SEQUENCE LISTING (8 sequences)

SEQ ID NO: 1 - DNA, 1200 bp, Mus sp.
acggagggaatagcctttcgattctgggtggtgcattggaagccccaggctctaaaacccccaacctactgactggtggccgagtatgcacccgactgctagctaggcagtgtcccaagaaccagtagccaaatgtcttggcctcagttttcccggtgacacctggaaagtgaccctgccattagtagaggctcaggtcagggccccgcctctcctgggcggcctctgccctagcccgccctgccgctcctcctctccgcaggctcgctcccacggtccccgaggtgggcgggtgagcccaggatgacggctgtagaaccccggcctgactcgccctcgcccccgcgccgggcctgggcttccctagcccagctcgcacccgggggccgtcggagccgccgcgcgcccagctctacgcgcctggccctccccacgcgggcgtccccgactcccgcgcgcgctcaggctcccagttgggaaccaaggagggggaggatgggggggggggtgtgcgccgacccggaaacgccatataaggagcaggaaggatcccccgccggaacagaccttatttgggcagcgccttatatggagtggcccaatatggccctgccgcttccggctctgggaggaggggcgagcgggggttggggcgggggcaagctgggaactccaggcgcctggcccgggaggccactgctgctgttccaatactaggctttccaggagcctgagcgctcgcgatgccggagcgggtcgcagggtggaggtgcccaccactcttggatgggagggcttcacgtcactccgggtcctcccggccggtccttccatattagggcttcctgcttcccatatatggccatgtacgtcacggcggaggcgggcccgtgctgttccagacccttgaaatagaggccgattcggggagtcgcgagagatcccagcgcgcagaacttggggagccgccgccgcgattcgccgccgccgccagcttccgccgccgcaagatcggcccctgccccagcctccgcggcagccctgcgtccaccacgggccgcggctaccgccagcctgggggcccacctacactccccgcagtgtgcccctgcaccccgcatgtaacccggccaacccccggcgagtgtgccctcagtagcttcggccccgggctgcgcccaccacccaacatcagttctc

SEQ ID NO: 2 - DNA, 684 bp, Homo sapiens
cggttcgctctcacggtccctgaggtgggcgggcgggcctggaggacagcgatagaaccccggcccgactcgccctcgcccccgctctgggtctgggcttccccagcctagttcacgcctaggagccgcctgagcagccgcgccaagcgccacacgccacgagccctccccgcctgggcgtccccggatcccgcgagcgctcgggctcccggcttggaaccagggaggagggagggagcgagggagcaaccagctcggaccggaatgcatatagagcaggaaggatcccccgccggaacaacccttatttgggcagcaccttatttggagtggccggatatggcccggcgcttccgcctctgggaggagggaagaaggcggagggaggggcaacgcgggaactccggagctgccggtcccggaggccccggcggcggctagagctctaggcttccccgaagctgggcgcctgggatgcgggccgggccgggccctagggtgcaggatggaggtgccgggcgctgtcggatggggggcttcacgtcactccgggtcctcccccggtcctgccatattagggcttctgcttcccatatatgccatgtacgtcacgacggaggcggacccgtgccgttccagacccttcaaatagaggcggatccggggagtcgcgagagatccagc

SEQ ID NO: 3 - DNA, 702 bp, Homo sapiens
cggttcgctctcacggtccctgaggtgggcgggcgggccctggatgacagcgatagaaccccggcccgactcgccctcgcccccgctctgggtctgggcttccccagcctagttcacgcctaggagccgcctgagcagccgcgcgcccagcgccacacgccacgagccctccccgcctgggcgtccccggatcccgcgagcgctcgggctcccggcttggaaccagggaggagggagggagcgagggagcaaccagctgcgacccggaaatgccatataagaagcaggaaggatcccccgccggaacaacccttatttgggcagcaccttatttggagtggcccgatatggcccggccgcttccggctctgggaggagggaagaaggcggagggaggggcaacgcgggaactccggagctgcgcgggtcccggaggccccggcggcggctagagctctaggcttccccgaagcctgggcgcctgggatgcgggcgcgggcgcgggccctagggtgcaggatggaggtgccgggcgctgtcggatggggggcttcacgtcactccgggtcctcccggccggtcctgccatattagggcttcctgcttcccatatatggccatgtacgtcacgacggaggcggacccgtgccgttccagacccttcaaatagaggcggatccggggagtcgcgagagatccagc

SEQ ID NO: 4 - DNA, 702 bp, Homo sapiens
cggttcgctctcacggtccctgaggtgggcgggcgggccctggatgacagcgatagaaccccggcccgactcgccctcgctatcgctctgggtctgggcttccccagcctagttcacgcctaggagccgcctgagcagccgcgcgcccagcgccacacgccacgagccctccccgcctgggcgtccccggatcccgcgagcgctcgggctcccggcttggaaccagggaggagggagggagcgagggagcaaccagctgcgacccggaaatgccatataagaagcaggaaggatcccccgccggaacaacccttatttgggcagcaccttatttggagtggcccgatatggcccggccgcttccggctctgggaggagggaagaaggcggagggaggggcaacgcgggaactccggagctgcgcgggtcccggaggccccggcggcggctagagctctaggcttccccgaagcctgggcgcctgggatgcgggcgcgggcgcgggccctagggtgcaggatggaggtgccgggcgctgtcggatggggggcttcacgtcactccgggtcctcccggccggtcctgccatattagggcttcctgcttcccatatatggccatgtacgtcacgacggaggcggacccgtgccgttccagacccttcaaatagaggcggatccggggagtcgcgagagatccagc

SEQ ID NO: 5 - DNA, 33 bp, Artificial Sequence
  Description of Artificial Sequence Primer
ggccacgcgtcgtcggttcgctctcacggtccc

SEQ ID NO: 6 - DNA, 30 bp, Artificial Sequence
  Description of Artificial Sequence Primer
gcagctcgaggctggatctctcgcgactcc

SEQ ID NO: 7 - DNA, 52 bp, Artificial Sequence
  Description of Artificial Sequence isolated SRE 5 of human Egr-1 promoter
aggctgcgacccggaaatgccatataagaagcaggaaggatccccccgccgg

SEQ ID NO: 8 - DNA, 52 bp, Artificial Sequence
  Description of Artificial Sequence isolated SRE 5 of human Egr-1 promoter (complementary strand)
agccggcggggggatccttcctgcttcttatatggcatttccgggtcgcagc

(19) United States
(12) Patent Application Publication
(10) Pub. No.: US 2003/0124554 A1
(43) Pub. Date: Jul. 3, 2003

(54) Screening method for compounds capable of modularing egr-1-regulated expression

(76) Inventors: Martin Braddock, St. Neots (GB); Callum Jeffrey Campbell, Pinner (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/148,055

(22) PCT Filed: Nov. 30, 2000
(86) PCT No.: PCT/EP00/11997

(51) Int. Cl. C12Q 1/68; G01N 33/53; G01N 33/567; A61K 31/00

(52) U.S. Cl. 435/6; 435/7.2; 514/1

(57) ABSTRACT

An Egr-1 expression system can be provided and used to screen for moieties capable of regulating Egr-1 expression. Such moieties can be used as medicaments or in drug development programs, particularly in the identification of drugs for wound healing.

The present invention relates, inter alia, to screening. It includes methods of screening for moieties useful as therapeutic agents or useful in the development of therapeutic agents via drug screening programs.

The healing of skin involves a wide range of cellular, molecular, physiological and biochemical events. During the healing process, cells migrate to wound sites where they proliferate and synthesise extracellular matrix components in order to reconstitute a tissue closely similar to the uninjured original. This activity is regulated by mediators secreted from the wound border cells such as platelet-derived growth factor (PDGF), epidermal growth factor (EGF), transforming growth factor (TGF) beta and other cytokines. Beneficial effects of these agents on cells has been demonstrated both in vitro and in vivo (reviewed by Moulin, Eur. J. Cell Biol. 68; 1-7, 1995), including benefit of administering PDGF in rat models of diabetes (Brown et al J. Surg. Res. 56; 562-570, 1994).

Over the last few years numerous growth factors have been shown to accelerate cell proliferation in vitro and to promote wound healing in animal models. TGF beta has received the greatest attention in the context of wound repair as it promotes cell proliferation, differentiation and matrix production. TGF beta administered either topically or systemically accelerates the rate of cutaneous wound repair in animal models. (Ashcroft et al Nature Medicine, 3; 1209-1215, 1997; Sporn and Roberts J. Cell Biol. 119;1017-1021, 1997; Beck et al J. Clin. Invest. 92; 2841-2849, 1993). Likewise PDGF has been reported to promote re-epithelialisation and revascularisation in ischemic tissue and diabetic animals (Uhl et al Langenbecks Archiv fur Chirurgie-Supplement-Kongressband 114; 705-708, 1997 and reviewed in Dirks and Bloemers Mol. Biol. Reports 22; 1-24, 1996).

The transcription factor Egr-1 is a potential regulator of over 30 genes and plays a role in growth, development and differentiation (reviewed in Liu et al Crit. Rev. Oncogenesis 7; 101-125, 1996; Khachigian and Collins Circ. Res. 81; 457-461, 1997). Egr-1 is induced upon injury to the vascular endothelium (e.g. Khachigian et al Science; 271, 1427-1431, 1996) and targets for transcriptional activation are numerous genes including epidermal growth factor (EGF), platelet-derived growth factor A (PDGF A), basic fibroblast growth factor (bFGF), induction of PDGF A, PDGF B, TGF beta, bFGF, uro-plasminogen activator (u-PA), tissue factor and insulin-like growth factor-2 (IGF-2).

The transcription complex that mediates vascular endothelial growth factor (VEGF) induction is dependent upon AP2 and not Egr-1 directly (Gille et al EMBO J 16; 750-759, 1997). However PDGF B directly upregulates VEGF expression (Finkenzeller Oncogene 15; 669-676, 1997). Transcription of VEGF mRNA is enhanced by a number of factors including PDGF B, bFGF, keratinocyte growth factor (KGF), EGF, tumour necrosis factor (TNF) alpha and TGF beta1. VEGF has been to promote re-endothelialisation in the balloon injured artery. Data obtained in rabbits demonstrated a clear VEGF driven passivation of metallic stents effecting an inhibition of in-stent neo-intima formation, a decrease in the occurrence of thrombotic occlusion, an acceleration of re-endothelialisation of the prosthesis and an increase in vasomotor activity (van Belle, E. et al, Bichem. Biophys. Res. Comm., 235; 311-316, 1997; van Belle, E. et al, J. Am. Coll. Cardiol., 29; 1371-1379, 1997; Asahara, T., et al, Circulation, 94; 3291-3302, 1997). NIH approval for a pilot study of VEGF to promote re-endothelialisation in humans was granted in 1996. In addition, HGF has also been shown to promote re-endothelialisation following balloon angioplasty in a rat model of carotid artery injury (Nakamura et al, Abstract 1681, American Heart Association Meeting; Dallas, 1998). In animal models, VEGF-driven passivation of metallic stents has been shown to inhibit neo-intima formation, accelerate re-endothelialisation and increase vasomotor activity (Asahara et al Circulation; 94, 3291-3302).

VEGF expression has been reported in healing wounds and psoriatic skin, both conditions in which TGF alpha and its ligand the EGF receptor (EGFr) are upregulated. Expression of EGF induces Egr-1 (Iwami et al Am. J. Physiol. 270; H2100-2107, 1996; Fang et al Calcified Tissue International 57; 450-455, 1995; J. Neuroscience Res. 36; 58-65, 1993). There is at present anecdotal evidence that Egr-1 may activate the expression of inter-cellular adhesion molecule-1 (ICAM-1) in phorbol ester stimulated B lymphocytes (Maltzman et al Mol. Cell. Biol. 16; 2283-2294, 1996) and may activate the expression of TNF alpha by virtue of the presence of an Egr-1 binding site in the TNF alpha promoter (Kramer et al Biochim. Biophys. Acta 1219; 413-421, 1994). Finally, Egr-1 knock out mice are infertile and luteinizing hormone (LH) deficient (Lee et al, Science 273; 1219-1221, 1996) implying that the LH promoter may also be a target for Egr-1 activation.

Bone loading, mechanical stretch and fluid flow of osteoblast-like MC3T3E1 cells induces Egr-1 (Dolce et al Archs. Oral Biol. 41; 1101-1118, 1996; Ogata J. Cell Physiol. 170; 27-34, 1997) with concomitant activation of growth factors. Egr-1 expression predominates in the cartliage and bone of the developing mouse (McMahon et al Development 108; 281-287) and has been implicated in the regulation of growth and differentiation of osteoblastic cells (Chaudhary et al Mol. Cell. Biochem. 156; 69-77,1996). Egr-1 and the closely related zinc finger transcription factor wilm's Tumour 1 (WT1) have been implicated in the regulation of osteoclastogenesis (Kukita et al Endocrinology 138; 4384-4389, 1997) and both prostacyclin E2 (PGE2) and EGF are induced by Egr1 (Fang et al Calcified Tissue International 57; 450-455, 1995; Fang et al Prostoglandins, Leukotrienes and Essential Fatty Acids 54; 109-114, 1996). Vascular calcification is an actively regulated process similar to bone formation involving cells and factors known to be important in the regulation of bone metabolism (reviewed in Dermer et al Trends Cardiovasc. Med. 4; 45-49, 1994). Regulators of osteoblastogenesis and/or osteoclastogenesis may modulate the degree of vessel wall calcification.

Hypertrophic stimuli such as haemodynamic load and angiotensin II may be used to drive the production of Egr-1 dominant negative under the control of a myocyte specific promoter and have application in the treatment of heart failure.

Egr-1 is essential for Schwann cell expression of the p75 nerve growth factor (NGF) receptor (Nikam et al Mol. Cell. Neurosciences 6; 337-348, 1995). NGF induces Egr-1 expression with concomitant activation of growth factors (Kendall et al Brain Research. Molecular Brain Research. 25; 73-79, 1994; Kujubu et al Journal of Neuroscience Research 36; 58-65, 1993).

The stimulus from the cell surface to Egr-1 is mediated in part by the MAP kinase signalling cascade (e.g. Schwachtgen et al J. Clin. Invest. 101; 2540-2549, 1998). Egr-1 when delivered as a gene therapy controls expression of a variety of growth factors and cytokines. International patent application number PCT/GB99/01722 describes the use of the Egr-1 transcription factor DNA to promote angiogenesis, re-epithelialisation, collagen production and wound contraction in skin in healthy animal models of acute injury. In addition, Egr-1 has been shown to induce ectopic bone formation in rats.

The expression of Egr-1 can be regulated by a number of means. This may include activation of the MAPK pathway and by the G protein G alpha13 via the small G protein Rho (Prasad and Dhanasekaran Oncogene 18; 1639-1642, 1999), two signalling pathways which are stimulated following lipid binding to EDG receptors (Goetzl and An FASEB J. 12; 1589-1598, 1998). It has been shown that S1P can stimulate induction of Egr-1 and basic fibroblast growth factor via a MAPK-dependent mechanism in C6 glioma cells (Sato et al Molecular Pharmacology 55; 126-133, 1999). Another mechanism which may increase the activity or production of Egr-1 includes modulating the interplay of Egr-1 with its natural repressor proteins known as the NABS. The NAB proteins NAB1 and NAB2 (NGFI-A binding corepressors) interact with the conserved R1 domain of the Egr-1 and Egr-2 trans-activators (Svaren et al EMBO J., 17; 6010-6019, 1998). It has previously been shown that NAB2 will repress the NGF-induced differentiation of PC12 cells (Qu,Z. et al J. Cell Biol. 142; 1075-1082, 1998) and Egr-1 mediated-activation of basic FGF (Svaren et al EMBO J., 17; 6010-6019, 1998). International patent application number PCT/GB99/02199 describes the use of NAB2 to repress Egr-1 mediated production and secretion of PDGF-AB, TGF beta, HGF and VEGF, Egr-1 driven angiogenesis and to repress endogenous production and secretion of PDGF-B.

According to the present invention there is provided a method of screening comprising providing an expression system that is regulated by Egr-1 and analysing the effect of a moiety being screened on expression from said system.

Expression may be analysed qualitatively and/or quantitatively.

The expression system preferably comprises a nucleic acid molecule having at least one Egr-1 binding site. More preferably, at least two or at least three such sites are present.

The expression system may include an Egr-1 dependent promoter. The term “Egr-1 dependent promoter” indicates that Egr-1 affects the level of transcription from the promoter. Thus even though transcription may occur from said promoter in the absence of Egr-1, when Egr-1 is added to a given transcription system the level of transcription will change—i.e. it will increase or decrease. Preferably it will increase.

Desirably the Egr-1 binding sites have one of the following sequences:

5′-GCG(T/G)GGGGCG-3′ or 5′-GCCGGGGGCG-3′ or 5′-TCC(T/A)CCTCCTCC-3′

It is however important to note that any Egr-1 binding site(s) capable of conferring Egr-1 dependency upon a promoter can be used. Egr-1 dependency can easily be assayed by comparing transcription or translation in the presence of such a site/sites with that in the absence of such a site/sites.

The human Egr-1 promoter is known and is disclosed in international patent application number PCT/GB99/01722, as well as in FIG. 7 of the present application. It comprises an Egr-1 binding site that may be involved in autoregulation. If desired, this promoter may be used in an expression system for screening according to the present invention. A variant of this promoter that is also regulated by Egr-1 may also be used in the present invention. Preferred variants will have substantial sequence identity with the GW SEQ shown in FIG. 7. Variants can include naturally occurring Egr-1 promoters from non-human mammals, e.g. mouse and rat. Allelic variants (whether from human or non-human animals are also included.) Desirably variants of the human Egr-1 promoter comprise one or more of the boxed regions shown in Figure for GW SEQ. (The EBS and TATA boxes are most preferred.) Variants may include one or more of the underlined nucleotides shown in FIG. 7.

It is however important to note that the human Egr-1 promoter or a variant thereof need not be used in the present invention. Any promoter that is regulatable by Egr-1 can be used. Suitable known promoters include: tissue factor, PDGF A and PDGF B promoters.

Furthermore, additional promoters can be constructed. This can be done by modifying known promoters that are not Egr-1 dependent—e.g. by providing them with one or more Egr-1 binding sites and then selecting modified promoters that are regulated by Egr-1.

If necessary, Egr-1 may be added to an expression system of the present invention. Alternatively (and more preferably) the expression system may itself provide Egr-1. (Many different cell types are known to express Egr-1.) Thus Egr-1 may be provided by stimulation of endogenous Egr-1 expression in addition to a heterologous polypeptide that may be expressed using a promoter that is regulatable by Egr-1.

An expression system for use in the present invention may be a cell-based expression system or a cell-free expression system. Expression can be of any detectable substance. The term “expression” therefore includes not only expression of polypeptides (products of translation) but also of RNA (products of transcription).

RNA expression can be monitored by using probes (which may be labeled), for example. Preferred probes hybridise to RNA of interest under conditions of moderate or high stringency. Hybridisation conditions are discussed in detail at pp 1.101-1.110 and 11.45-11.61 of Sambrook et al [Molecular Cloning, 2nd Edition, Cold Spring Harbor Laboratory Press (1989)]. One example of hybridisation conditions that can be used involves using a pre-washing solution of 5×SSC, 0.5% SDS, 1.0 mM EDTA (pH 8.0) and attempting hybridisation overnight at 55° C. using 5×SSC. However, there are many other possibilities. Some of these are listed in Table 1 of WO98/45435, for example. (See especially the conditions set out under A-F of that table and, less preferably those listed under G to L or M to R.)

Polypeptide expression can be monitored e.g. by using antibodies or other binding agents (such as antibody fragments or derivatives, lectins, etc.) The binding agents may be labeled if desired. Monitoring for a particular activity of the polypeptide being expressed (e.g. an enzymatic activity) may also/alternatively be performed.

An expression system for use in the present invention may comprise a coding sequence that occurs in nature in association with the Egr-1 dependent promoter. Alternatively, a heterologous coding sequence may be provided that is operably linked with the Egr-1 dependent promoter. This can be provided using genetic engineering techniques. Such techniques are disclosed in standard text-books, such as in Sambrook et al [Molecular Cloning 2nd Edition, Cold Spring Harbor Laboratory Press (1989)] and in Old & Primrose [Principles of Gene Manipulation 5th Edition, Blackwell Scientific Publications (1994)].

A preferred expression system of the present invention uses a reporter gene. The reporter gene can be chosen to express a substance that can be detected by a standard assay procedure. For example, the reporter gene may encode an enzyme that can bring about a reaction leading to a colour change or a change in fluorescence or luminescence. One such enzyme is luciferase. This can be detected using a luminometer and a commercially available detection system (e.g. the “Steady-Glo” luciferase detection system available from Promega.)

A screening method of the present invention may include a control step, whereby expression from the expression system is compared with expression from a system that is not regulated by Egr-1 or that is regulated to a lesser extent by Egr-1. Thus it may be compared with expression from a system that does not include effective Egr-1 binding sites (e.g. a system in which Egr-1 binding sites have been inactivated by mutation—i.e. by deletion, insertion or replacement of nucleotides)

A screening method of the present invention may include other controls. For example, expression in the presence of a moiety being screened may be compared with expression in the presence of a substance known to increase Egr-1 activity or expression (e.g. EGF). Alternatively, expression in the presence of a moiety being screened may be compared with expression in the presence of a substance known to reduce Egr-1 activity or expression (e.g. NAB1 or NAB2).

A screening method of the present invention may include a step of analysing whether or not a moiety being screened binds to a nucleic acid present in the expression system, or to a specific part thereof (e.g. to an Egr-1 binding site), or to Egr-1 itself.

In a preferred screening method of the present invention, screening is performed to identify moieties that interact directly with an Egr-1 binding site or with Egr-1 itself.

Single moieties can be screened using the methods of the present invention. Alternatively, libraries comprising a plurality of moieties can be screened (wholly or partially). Libraries of moieties for screening are available to pharmaceutical companies and are available commercially. Libraries can also be provided de novo for screening. Many different types of library can be used. A library may consist of naturally occurring moieties (e.g. biological moieties), of non-naturally occurring moieties, or of a mixture of naturally occurring and non-naturally occurring moieties. The moieties may provided in solution, on beads, on chips (see e.g. Fodor (1993) Nature 364:555-556), on bacteria (see e.g. U.S. Pat. No. 5,223,409), on spores (see e.g. U.S. Pat. No. 5,223,409), on phage (see e.g. Scott and Smith (1990) Science 249:386-90 and U.S. Pat. No. 5,223,409), etc.

In addition to the screening methods of the present invention, the present invention includes moieties identified by screening. If desired, such moieties may be provided in substantially pure form or in isolated form. They may be useful in medicine and may be provided in the form of a pharmaceutically acceptable composition, optionally together with a pharmaceutically acceptable carrier. They can be administered via any suitable route of administration and at any appropriate dosage.

Moieties capable of increasing Egr-1 activity or expression are within the scope of the present invention. They can be used to treat conditions in respect of which Egr-1 is beneficial, including: wounds (e.g.wound healing) and associated conditions, such as in the treatment of dermal ulcers arising from ischaemia and neuropathy associated with diabetes, peripheral arterial occlusive disease, deep vein thrombosis, chronic venous insufficiency and pressure sores, reduction of post-operative scarring associated with, for example, cataracts, skin graft procedures burns, psoriasis, tissue engineering; acceleration of tissue remodelling and regeneration; hard tissue repair, for example bone; soft tissue repair, for example tendon, ligament, muscle, the promotion of angiogenesis, re-endothelialisation following percutaneous trans-luminal coronary angioplasty, inhibition of left ventricular cardiac hypertrophy, modulation of vessel wall calcification and the promotion of neuroregeneration, promotion of tissue repair in tissue destructive diseases such as chronic obstructive pulmonary disease.

Preferred moieties that are capable of increasing Egr-1 activity or expression are those that are useful in treating wounds (e.g.wound healing). Screens for such moieties are therefore preferred screens of the present invention.

Moieties capable of decreasing Egr-1 activity or expression are also within the scope of the present invention. They can be used to treat various conditions—especially conditions involving wounds (e.g wound healing) or cell proliferation. In particular, they can be used to treat conditions in respect of which it is beneficial to reduce the level of Egr-1 By way of example, such moieties can be used in the treatment of the following: reducing scarring during wound healing (e.g. reducing the incidence of hypertrophic/keloid scar formation and occular scarring after, for example glaucoma surgery), restenosis following percutaneous trans-luminal coronary angioplasty, modulation of vessel wall calcification, treatment of cancer or other cell proliferative disorders, the inhibition of fibrotic conditions, for example, pulmonary and liver fibrosis, and the prevention of alopecia.

Preferred moieties that are capable of decreasing Egr-1 activity or expression are those that are useful in reducing scarring during wound healing. Screens for such moieties are therefore preferred screens of the present invention.

The present invention includes within its scope the use of a moiety identified by screening in the manufacture of a medicament for treating any of the above-mentioned conditions.

Moieties of the present invention may be used in a drug development program. Thus they may be useful in providing an initial structure that may (if necessary) be modified to produce a drug that is approved for use for a given therapy. Such a drug development program is within the scope of the present invention, as is a drug developed from it and medical uses thereof. The development program will typically involve performing a screen, taking a moiety identified by screening, modifying it and determining the effect(s) of modification (e.g. the effect(s) on activity and/or toxicity).

### FIGURES

FIGS. 1a through to 10, are as set out in international patent application PCT/GB99/01722 and are provided for reference purposes to show how various Egr-1 activities can be assayed and also to provide background information regarding the Egr-1 promoter.

FIGS. 1a and b show Egr-1 expression of VEGF;

FIGS. 1c and d show Egr-1 expression of TGF-B1;

FIGS. 1e and f show Egr-1 expression of PDGF A;

FIG. 2a shows the effect of Egr-1 on rat excisional wound contraction;

FIG. 2b shows the effect of Egr-1 DNA transfection on the histology of healing rat excisional wounds;

FIG. 2c shows the effect of Egr-1 on collagen deposition on rat excisional wounds;

FIG. 2d shows the effect of Egr-1 on the angiogenic profile in rat excisional wounds using vWF immunostaining;

FIG. 3a shows the optimisation of lipid:DNA ratio (v/w) for transfection of pGL3 luciferase control plasmid into the angiogenesis co-culture system using Mirus TransIT (Cambridge Bioseciences);

FIG. 3b shows the effect of Egr-1 on angiogenesis;

FIG. 4a shows the samples for bone loading using western blot analysis;

FIG. 4b shows the western blot analysis of Egr-1 protein in human TE85 bome cells exposed to load;

FIG. 4c shows an ELISA analysis of PDGF BB produced from TE85 bone cells after exposure to load;

FIG. 4d shows the detection of VEGF and TGF-B1 after transfection of CMV-TGF-B1 in ROS cells;

FIG. 4e shows detection of VEGF and TGF-B1 after transfection of CMV-TGF-B1 in MC3tE1 cells;

FIG. 5 shows effect of Egr-1 on alkaline phosphatase levels in a rodent model of ectopic bone formation;

FIG. 6a shows anti-Egr-1 antibody staining of human smooth muscle cells transfected with CMV Egr-1;

FIG. 6b shows anti-Egr-1 antibody staining of porcine smooth muscle cells transfected with CMV Egr-1 DNA;

FIG. 6c shows optimisation of transfection of pGL3 luciferase control in human SMC by Fugene;

FIG. 6d shows optimisation of transfection of pGL3 luciferase control in porcine SMC by Fugene;

FIG. 6e shows activation of VEGF production/secretion by transfection of CMV-Egr-1 into human SMC;

FIG. 6f shows activation of HGF production/secretion by transfection of CMV-Egr-1 into human SMC;

FIG. 6g shows activation of PDGF production/secretion by transfection of CMV-Egr-1 into human SMC;

FIG. 6h shows immunostaining of Egr-1 protein in vessel wall pre and post injury;

FIG. 7 shows a comparison of two nucleotide sequences indicated as GW SEQ and ON SEQ respectively. ON SEQ is the published early growth response-1 promoter (Sakamoto et al Oncogene 6; 867-871, 1991), and GW SEQ is a sequence in accordance with the invention, which contains a number of base insertions/deletions as shown and substitutions (bold-underlined).

FIG. 8 shows a variant of the sequence shown in FIG. 7, which variant has a modified EBS region Mutation in the Egr-1 binding site (EBS) is shown in bold-underline.

FIG. 9 shows the published 5′ upstream sequence of mouse Egr-1 gene (Morris, Nucleic Acids Research, 16:8835-8846). The nucleotides are numbered from the cap site=+1. Putative TATA and CCAAT elements are boxed. Potential regulatory elements are underlined and indicated in the figure. Dotted underline shows position of 29-mer used for primer extension studies;

FIG. 10 shows activation of SRE5 by transient transfection of pFA-MEK1.

### EXAMPLES

Examples 1 to 7 below are taken from international patent application number PCT/GB99/01722 and are provided for reference purposes. Example 8 relates to the present invention.

### Example 1-7

Examples 1 and 2 describe gene gun delivery of p-galactosidase and Egr-1 expression plasmid DNAs, complexed to gold particles, to rodent skin.

a) Preparation of Tubing Station

The tubing preparation equipment was set up in a sterile air laminar flow cabinet, and swabbed with 70% I.M.S. and air dried in the cabinet. Gas line tubing from the nitrogen cylinder to the tubing preparation equipment was autoclaved and a Gelman in-line 0.2 μm filter attached. The autoclaved tubing was connected to the gas inlet on the preparation equipment by a luer lock connector, and gas allowed to flow through at 0.2 litres/minute to dry the tubing completely.

The particle delivery tubing was attached to the preparation equipment and gas allowed to flow through as above to completely dry the interior of the tubing. Any residual moisture in the tubing will result in a poor or uneven attachment of the gold particles to the tubing walls and may adversely affect the outcome of any experiment.

b) DNA-gold Microcarrier Bead Preparation:

Gold beads of 1.0 μm were obtained from Bio-Rad UK. An aliquot of gold beads (53 mg) was weighed out into a microfuge tube, and 100 μl of 0.05M spermidine was added and the tube vortexed gently.

100 μl of DNA solution containing 100-120 μg of plasmid DNA expressing either Egr-1 or β-galactosidase was added followed by 100 μl of 1M CaCl₂ added dropwise while vortexing. This mixture was left to stand for 10 minutes at room temperature then spun down. The supernatant was removed and the gold pellet was washed three times in absolute EtOH.

The gold particles were finally resuspended in absolute ethanol containing 0.1 mg/ml polyvinyl pyrrolidone (PVP).

Estimation of coating efficiency of DNA to gold microcarriers, and release in aqueous solution: All samples (starting material, post-precipitation supernatant following DNA/gold complex formation, and eluted material) were assayed for DNA in a “GeneQuant” (Pharmacia). The residual post-precipitation DNA gave a measure of unbound material, and the ratio of bound: starting material was deemed to be the coating efficiency.

c) Loading the DNA/microcarrier Suspension into the Gold Delivery Tubing:

The gold particle suspension in ethanol/PVP was then loaded into the delivery tubing using a syringe, and the suspension allowed to stand for 3-5 minutes in the tubing. During this time the particles precipitated on the inner face of the tubing allowing the ethanol to be removed by the syringe. When the ethanol had been removed, the tubing was rotated to distribute the gold particles evenly on the inner face of the tubing. After 2-3 minutes rotation, nitrogen gas was passed through the tubing at a rate of 0.1 litres/minutes to remove residual ethanol and allow the gold particles to adhere. After 10 minutes, the tubing was removed, cut into appropriate lengths using the cutter provided (Bio-Rad UK), and the cut tubing was loaded into the gene gun.

Egr-1 expression and activity was determined using standard immunohistochemistry with commercially available antibody preparations for detection of Egr-1 (Santa Cruz), and Egr-1 target gene products (Santa Cruz or R&D systems) and expression was monitored from 1-7 days. The negative control was null DNA.

### Example 1

Delivery of Egr-1 DNA to Unwounded Rodent Skin

1.1 Methods

An expression plasmid comprising the Egr-1 cDNA driven from the human cytomegalovirus promoter (hCMV; Houston et al, Arterioscler. Thromb. Vasc. Biol., 19; 281-289, 1999) was delivered to the backs of unwounded mice via gene gun mediated particle delivery. Gold/DNA complexes were prepared as described above and 0.5-1.0 μg DNA was delivered per animal using a gene gun pressure of 350 psi and a gold particle size of 1.6 microns. Animals were sacrificed at day 0, 1, 2 and 6 days post delivery of DNA and the skin was embedded in OCT and snap frozen in dry ice/hexane. Sections were prepared at 0.7 μm and Egr-1 target growth factors examined by immunostaining using antibodies directed against VEGF, PDGF A, TGFβ and Egr-1.

1.2 Results

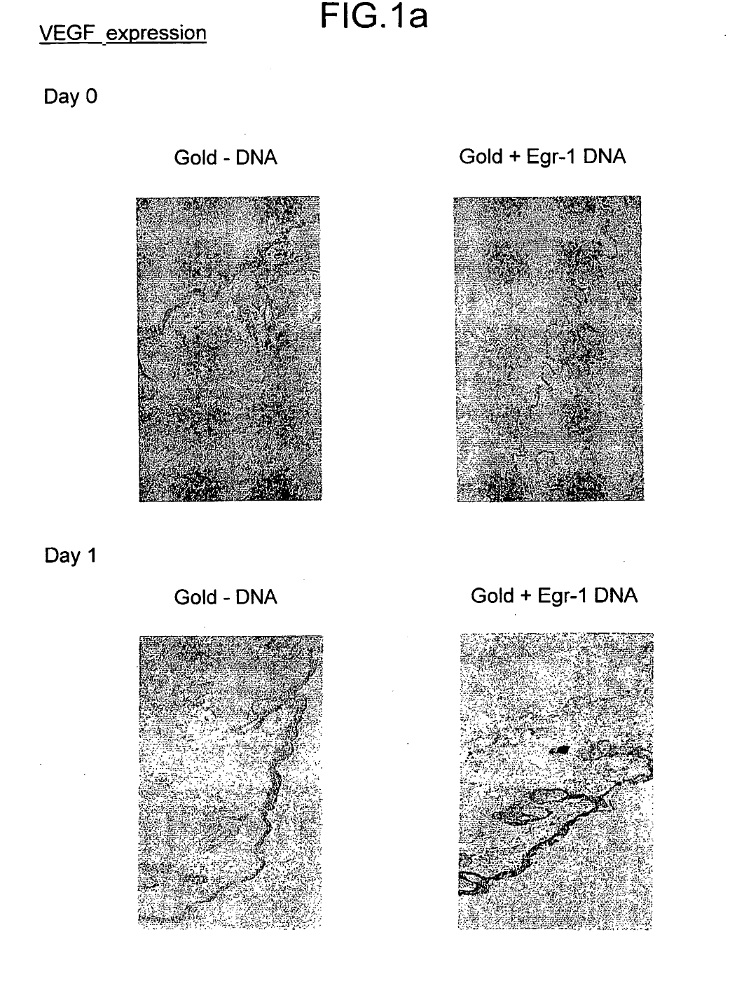

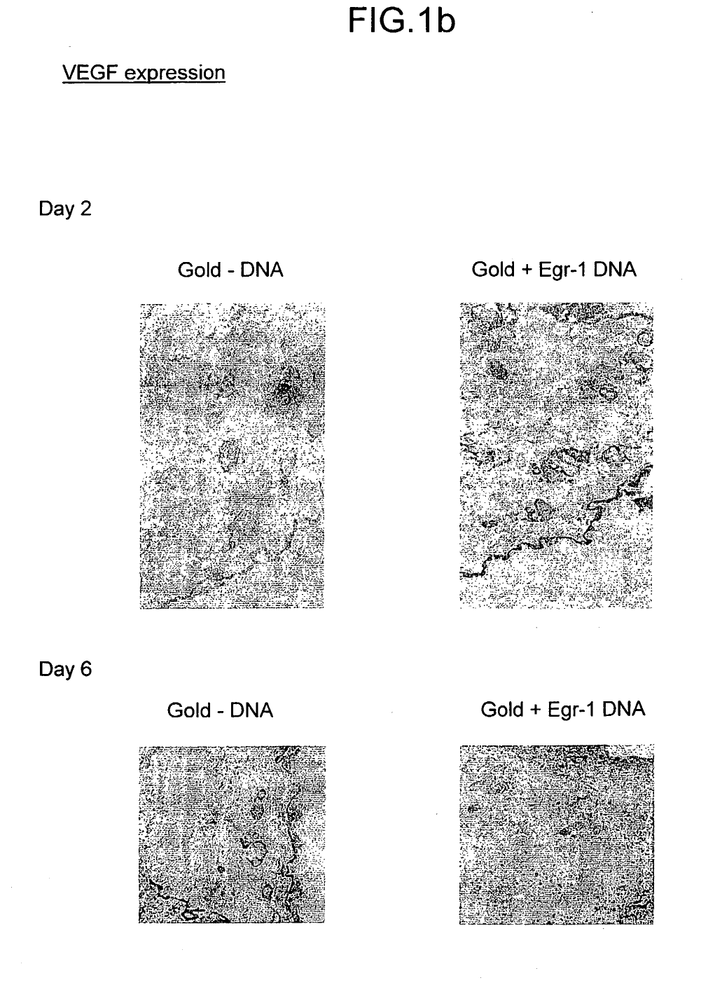

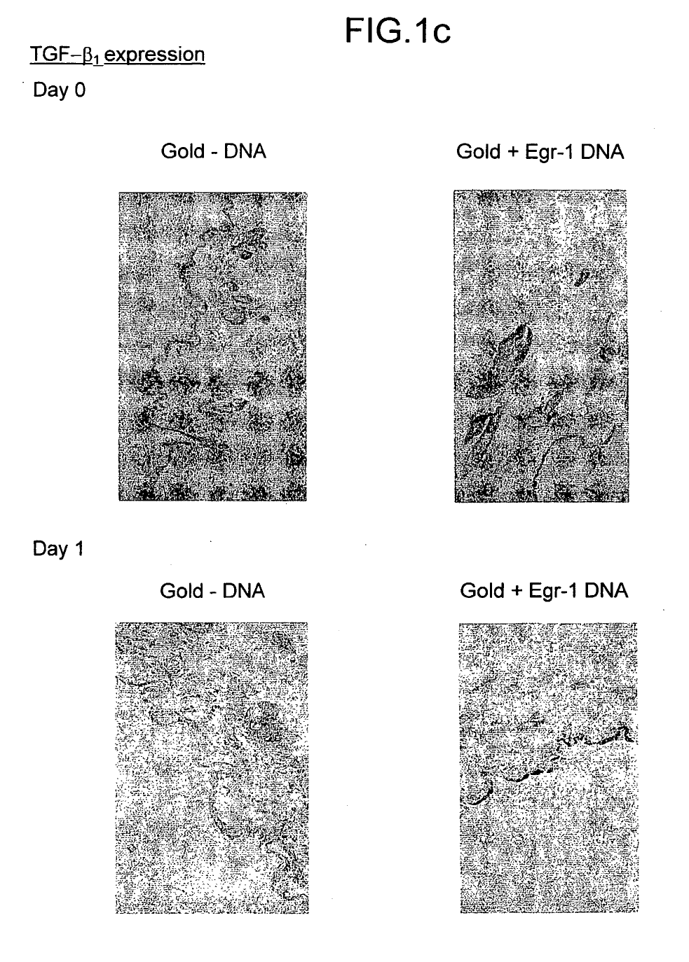

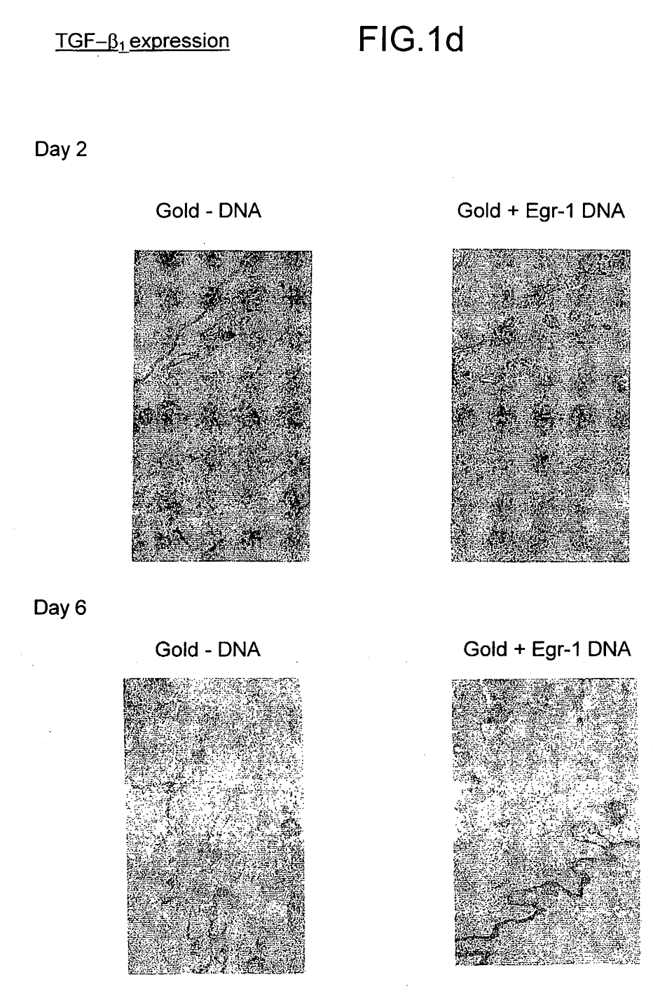

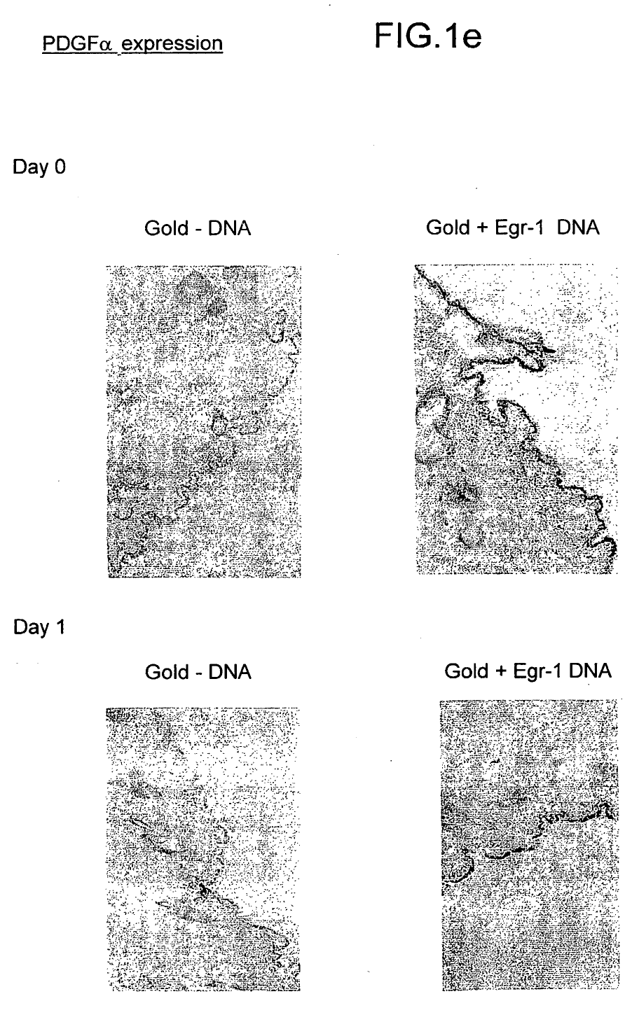

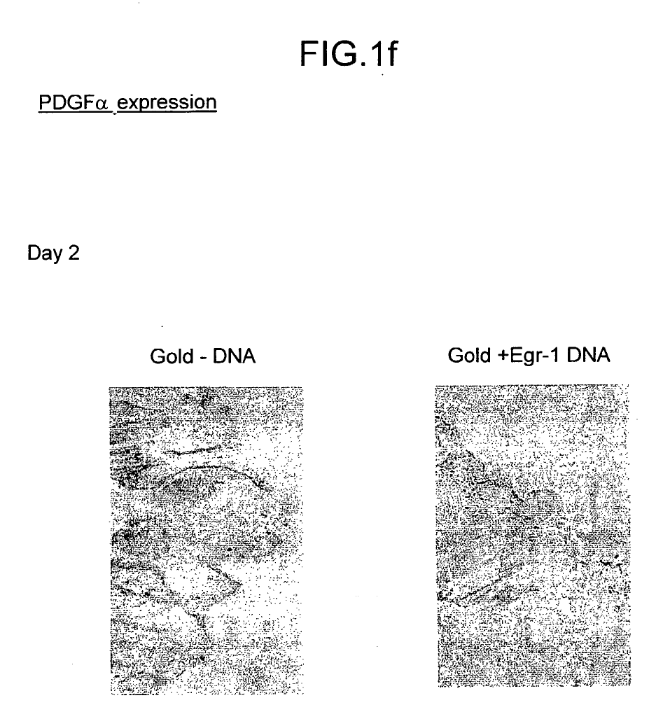

Immunohistochemical data is shown for Egr-1 activation of VEGF (FIGS. 1a. and 1b.), TGFβ (FIGS. 1c. and 1d.) PDGF A (FIGS. 1e. and 1f.). Results show dramatic upregulation of VEGF protein at days 1 and 2, declining at day 6, upregulation of TGFb at day 6 but not at days 1 and 2 and rapid up-regulation of PDGF A at 2 hrs post Egr-1 DNA delivery (designated day 0).

1.3 Conclusion

These data confirm that Egr-1 can activate the expression of target growth factors in vivo, some of which are described herein. These data illustrate that Egr-1 activation of growth factors occurs over a temporally separate timescale.

Having confirmed activation of Egr-1 target genes using unwounded rodent skin(Example 1), Egr-1 and β-galactosidase gene gun delivery of excisional rat wounds was performed to assess the effect of Egr-1 on the rate of healing.

### Example 2

Use of Egr-1 Transcription Factor to Promote Wound Repair in Rodents

2.1 Methods

2.1.1 Plasmid Constructs:

The expression plasmids used in this study were CMV driven β-galactosidase and CMV driven Egr-1 (Houston et al, Arterioscler. Thromb. Vasc. Biol, 19; 281-289, 1999). Plasmids were propagated in Eschericia coli XL-2 Blue MR and DNA was prepared using Qiagen maxi kits.

2.1.2 Particle Mediated Gene Transfer:

Eighteen male Sprague Dawley rats weighing 250 g were anaesthetised under isoflorane in a 2:1 mixture of oxygen/nitrous oxide. Two sites of transfection (8 cm from the skull, 1.5 cm either side of the spine) on the rat dorsum were prepared by firstly clipping the pelt then shaving with a razor. Two transfections were carried out per wound site, 8 mm apart, by accelerating plasmid/gold complexes of either Egr-1 or β-galactosidase into the skin at 350 psi. Total amount of DNA was not less than 1.7 μg per transfection (equalling 3.4 μg per wound).

2.1.3 Excisional Wound Healing Model:

Twenty-four hours post transfection animals were anaesthetised and 2 full thickness excisional wounds made (8 mm diameter) using a biopsy blade at the exact sites of transfection (see below). Immediately after wounding each wound was captured using camera/video set up and the animals allowed to recover from anesthesia. At 2, 4 and 6 days post wounding 6 animals were killed and each wound captured again using the same camera/video set up. Following capture, the wounds were dissected out and harvested for routine histology and immunohistochemistry.

2.1.4 Healing Analysis:

i) Macroscopic Assessment

Wound area was determined using image analysis and the healing expressed as a increase in percent of original wound area. Statistical significances of differences between treated and control groups were evaluated using a paired Mann-Whitney test.

ii) Microscopic Assessment

Histological Analysis:

Each wound per time point after dissection was bisected horizontally. One half was placed in 4% paraformaldehyde for 24 hours and processed for wax histology. 5 μm sections from each wound were cut using a microtome and the sections stained with van Geison. Using this histological stain, key markers of wound healing was assessed including re-epithelialisation and collagen content and comparisons made between treated and control sections.

Immunocytochemistry:

Immunocytochemistry and image analysis was performed in order to quantify the differences seen using routine histology. Once frozen in OCT, the second half of each wound was sectioned at 7 μm using a cryotstat. Two sections from each wound were fixed in ice-cold acetone and fluorescent immunostaining was performed with primary's to collagen I and von Willebrand factor (vWF). Immediately after immunostaining each slide was placed under a fluorescent microscope and the wound area captured using a ×25 magnification. The image was integrated and a threshold set to minimise background. Area and intensity of staining was measured using image analysis and plotted as a graph. Statistical significant differences between treated and control groups were evaluated using a Man-Whitney nonparametric test.

2.2 Results

2.2.1 Effect of Egr-1 on Rat Excisional Wound Healing

(i) Wound Contraction:

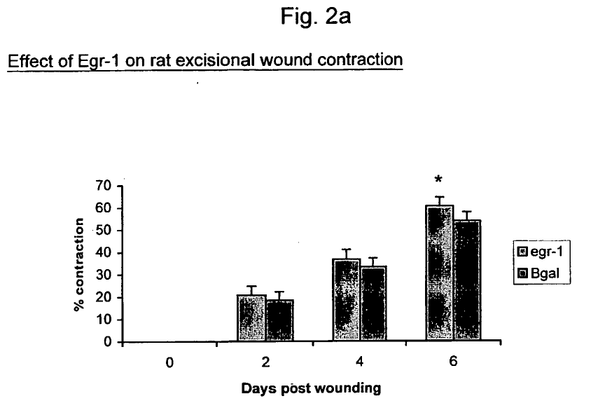

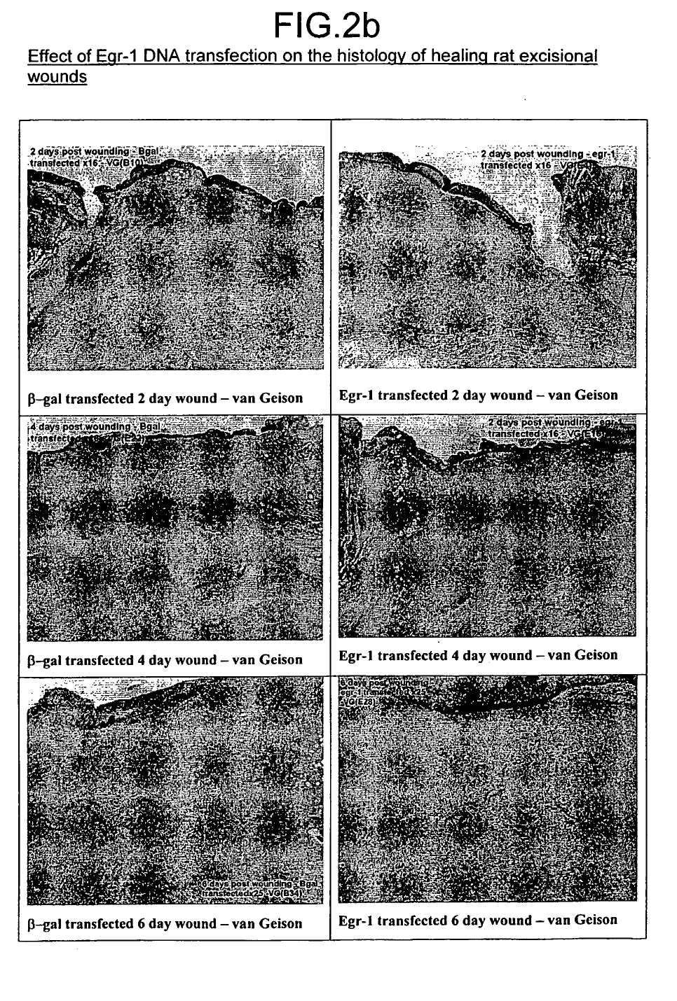

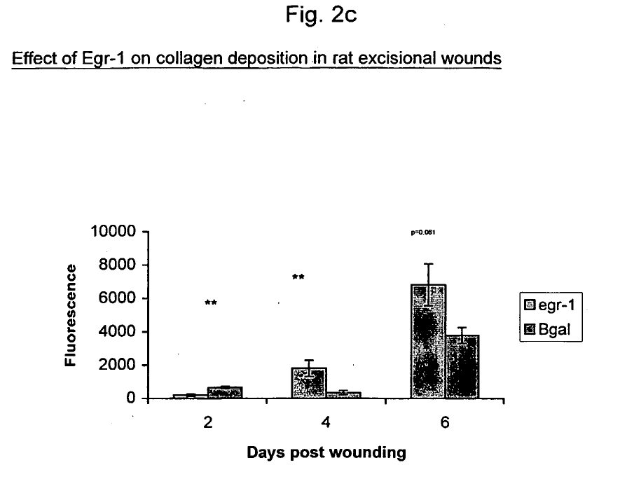

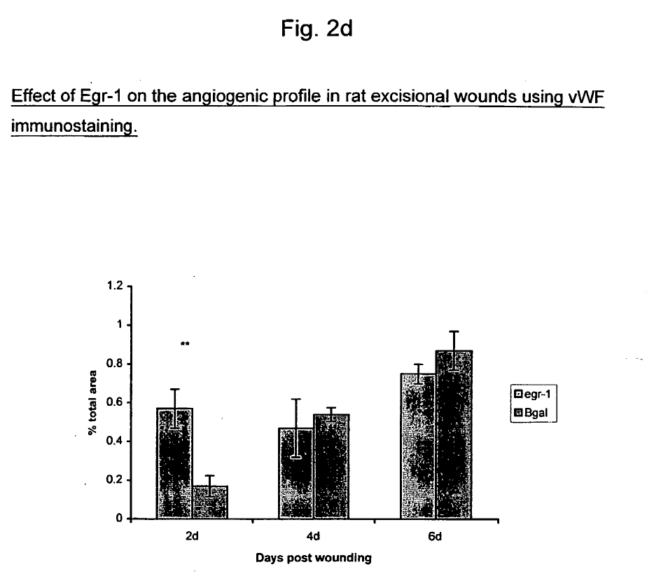

Full thickness rat dermal excisional wounds 8 mm in diameter contracted marginally faster in response to Egr-1 transfection compared to control (β-galactosidase) up to 6 days post wounding. Statistically significant enhancements of contraction (p<0.05) occurred at 6 days post wounding where Egr-1 treated wounds contracted to an area 7% smaller than control (FIG. 2a.).

(ii) Histological Analysis:

Wounds sections stained with van Gieson showed marked differences in the histology of wounds at 4 and 6 days post wounding. At 2 days post wounding there was little difference between Egr-1 and β-galactosidase transfected wounds. Both treatments showed mononuclear cells at the wound site indicating the early inflammatory response, with early scab formation, but no re-epithelialisation. At 4 days post wounding re-epithelialisation had still not commenced, however wounds transfected with Egr-1 had more collagen within the wound site compared to β-galactosidase. At 6 days post wounding wounds treated with Egr-1 had a more mature granulation tissue showing markedly more collagen within the wound site compared to β-galactosidase, to an extent where clear thick collagen fibres could be seen. Re-epithelialsation was complete in 50% of wounds treated with Egr-1 compared to 0% in β-galactosidase. Histologically Egr-1 treated wounds showed accelerated healing compare to β-galactosidase (FIG. 2b.).

(iii) Quantification of the Effect of Egr-1 on Collagen Deposition Using Immunohistochemistry and Image Analysis:

Collagen I immunostaining was performed on 7 μm crytosections of wounds treated with Egr-1 or β-galactosidase and the staining quantified using image analysis. Wounds treated with β-galactosidase had significantly more collagen at 2 days post wounding compared to Egr-1. At 4 and 6 days post wounding Egr-1 transfected wounds had more collagen deposition than control β-galactosidase) which confirms the findings seen using routine wax histology. Egr-1 transfection increased the amount of collagen deposition at 4 and 6 days post wounding (FIG. 2c.).

(iv) Quantification of the Effect of Egr-1 on Angiogenesis Using Immunohistochemistry and Image Analysis:

Angiogenesis was quantified using von Willebrand factor immunostaining on wound cryosections and image analysis to measure the area of positive staining within the wound site. At 2 days post wounding Egr-1 transfected wounds had significantly (p<0.01) more new blood vessels compared to control β-galactosidase). At 4 and 6 days post wounding both Egr-1 and β-galactosidase transfected wounds had similar levels of angiogenesis. Transfection of Egr-1 expressing DNA promoted angiogenesis 2 days earlier than control (FIG. 2d.)

2.3 Conclusions

Egr-1 transfection of rat excisional wounds accelerated healing by increasing the rate of contraction, re-epithelialisation and collagen deposition. Egr-1 transfection also promoted angiogenesis at 2 days post wounding.

### Example 3

Use of Egr-1 Transcription Factor to Promote Angiogenesis

3.1 Methods

Egr-1 under the control of the hCMV promoter (Houston et al, Arterioscler. Thromb. Vasc. Biol., 19; 281-289, 1999) was transfected into a human cell co-culture system designed to measure angiogenesis in vitro. The angiogenesis kit (TCS Biologicals) was used as described according to the manufacturer's instructions using VEGF protein (2 ng/ml) and suramin (20 μM) as positive and negative controls respectively for angiogenesis.

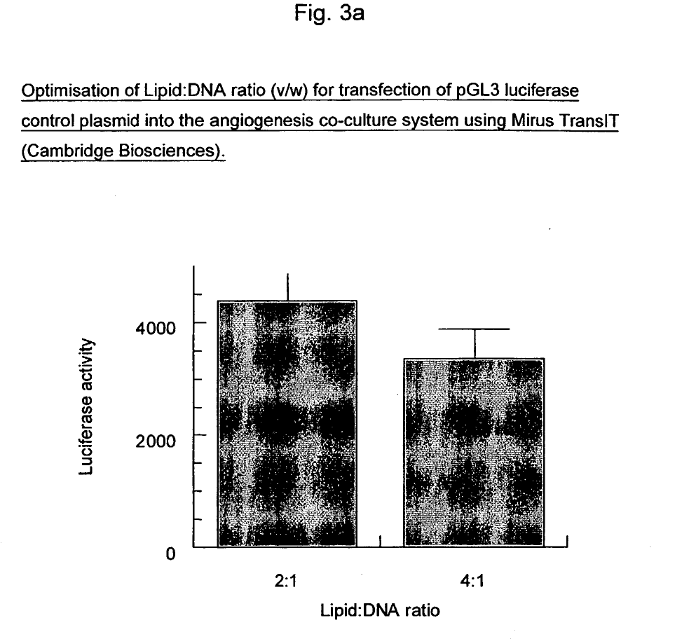

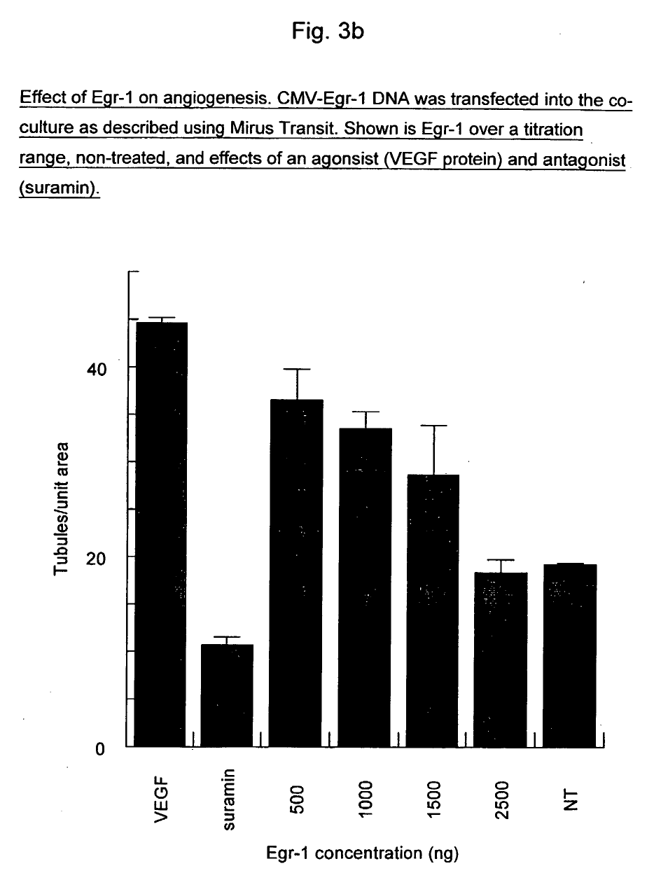

Optimisation of transfection in the co-culture system was performed using pGL3 control luciferase (Promega) with 1.0 μg and 0.5 μg CMV-β gal as a normalising plasmid for transfection control. Two ratios of lipid: DNA (v/w) were used; 2:1 and 4:1 (FIG. 3a.). CMV Egr-1 DNA was transfected at 0.5, 1.0, 1.5 and 2.5 μg per well in triplicate in a 24 well microtitre plate using Mirus Transit reagent (Cambridge Biosciences) at a ratio of 2:1 v/w DNA. VEGF protein positive control and suramin negative control was added to triplicate wells. After 11 days co-culture angiogenesis was determined by staining of cells for the endothelial cell marker PECAM-1 and visualisation using BCIP/NBT substrate.

Representative images of tubule formation using all four doses of Egr-1 expression plasmid together with VEGF (positive control) and suramin (negative control) were captured and processed by image analysis using Quantimet 600 image analyser and associated software.

3.2 Results

Angiogenesis as described by tubule formation visible under the light microscope was detectable after 11 days of co-culture. Angiogenic scoring is presented using image analysis as illustrated of the entire well and results are presented as tubules per unit area versus treatment (FIG. 3b.).

Decreased tubule formation (cells treated with suramin and increased tubule formation (cells treated with VEGF protein are shown. Egr-1 was shown to promote enhanced tubule formation in an inverse dose dependent fashion.

3.3 Conclusions

In the co-culture system, Egr-1 transcription factor expression is angiogenic. This supports and is supported by data from Example 1, whereby Egr-1 was shown to upregulate growth factor expression (e.g VEGF) when delivered by gene gun into mouse skin and data from Example 5, where transfection of Egr-1 was shown to increase the amount of VEGF produced in human vascular smooth muscle cells. The inverse dose response of Egr-1 as a pro-angiogenic stimulus is consistent with results obtained in Example 6 and with the notion that Egr-1 may down-regulate it's own production (Cao, X. et al, J. Biol. Chem., 268; 16949-16957, 1993; Schwachtgen, J.-L. et al, J. Clin. Invest., 101, 254-2549, 1998).

### Example 4

Use of Egr-1 Transcription Factor to Promote Osteogenesis in Vitro

4.1 Bone Loading and Determination of Growth Factors

4.1.1 Methods:

Cells used were TE85, a human osteosarcoma-derived osteoblast-like cell line. Sub-confluent cell layers were typsinised and resuspended in DMEM containing 10% foetal calf serum (FCS) and 1% penicillin-streptomycin (PS) antibiotics. The cell suspension was seeded onto the loading substrate (18×18 mm squares of tissue culture-treated plastic). Cells were left overnight to attach. Once attached the loading substrates and attached cells were transferred into flasks containing DMEM with 2% FCS and 1% PS for a further 24 hr prior to load stimulation.

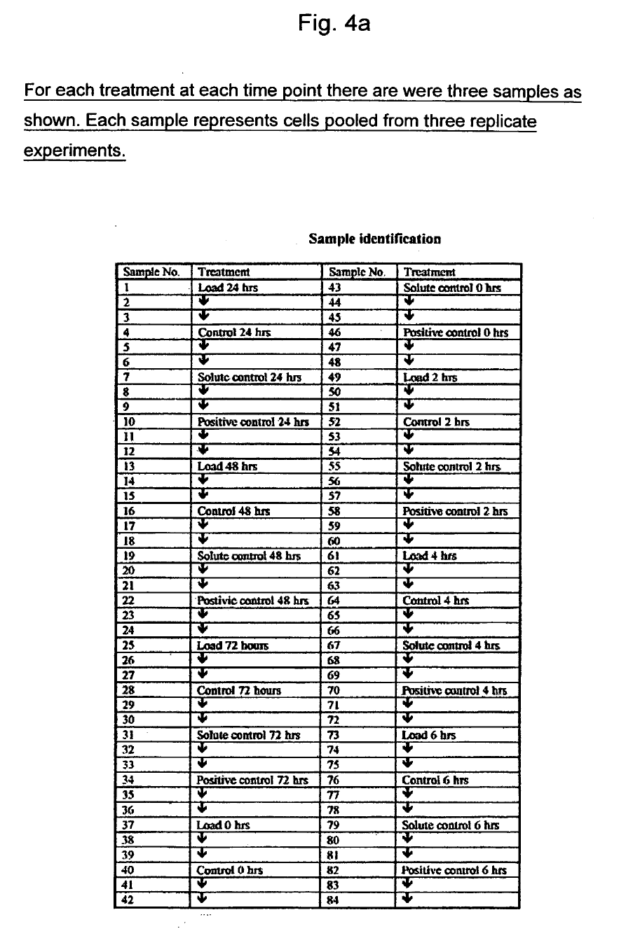

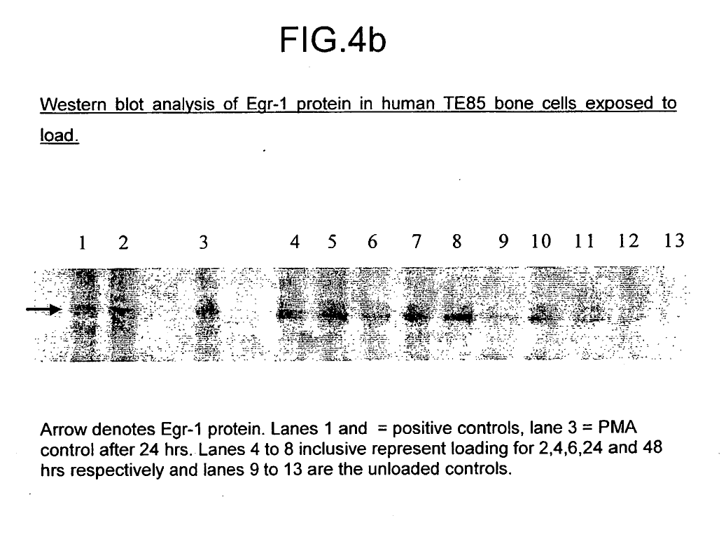

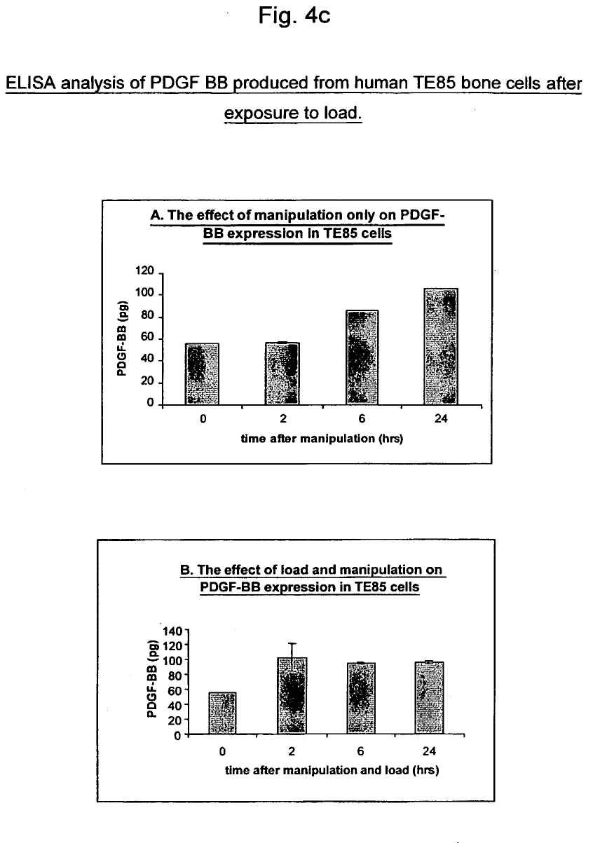

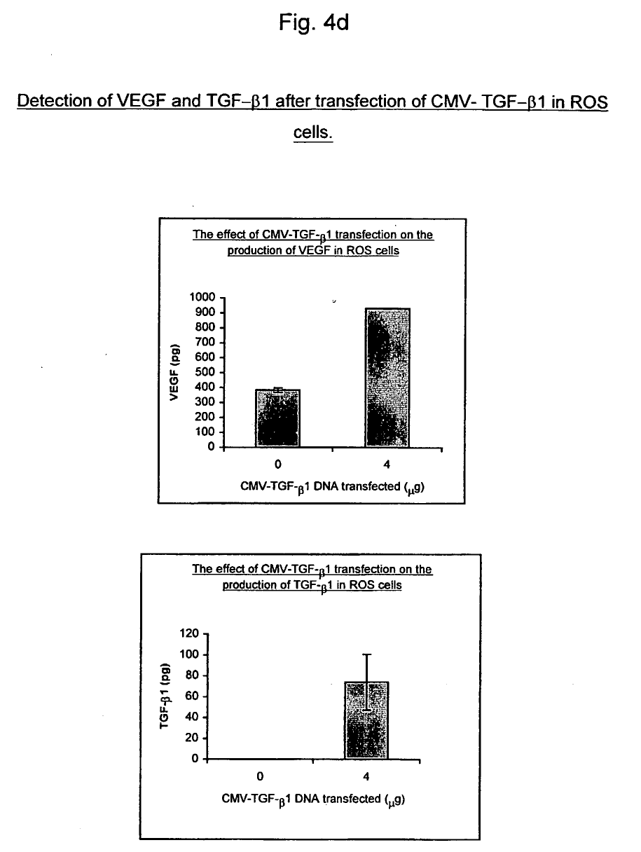

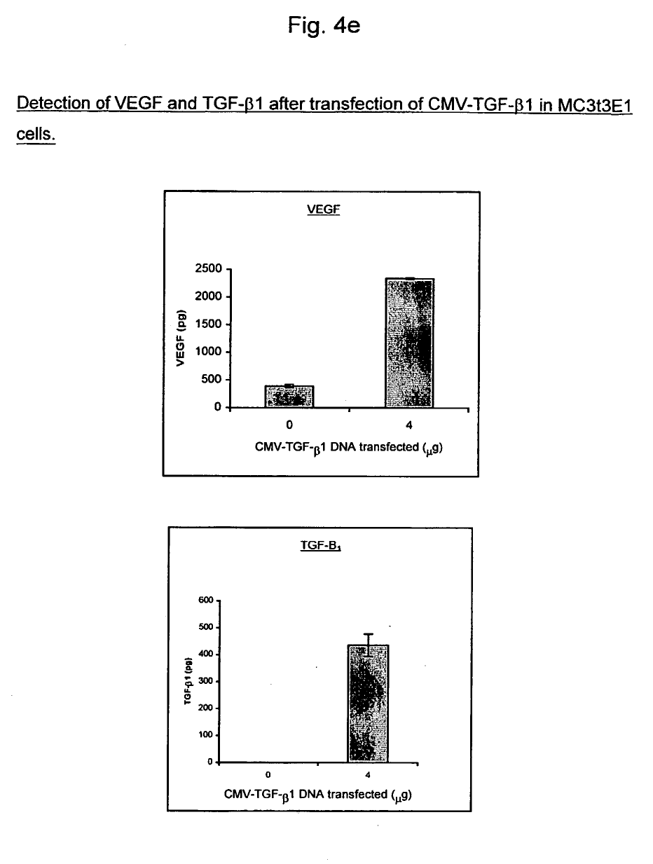

There were four sets of conditions for each time point described in FIG. 4a.:

[1]. Load (200 cycles of 2000 microstrain at 3232 microstrain per second).

[2]. Control (no load).

[3]. Positive control (100 ng/ml PMA for 1 hr).

[4]. Solute control.

For cell loading, cells were aseptically transferred from standard tissue culture conditions into the loading chamber. The duration of loading the cells in the chamber was 4 minutes. After loading, cells were then returned to their former culture conditions. Control treated cells were treated in exactly the same way except that no load was applied to the chamber.

Results were analysed by two different methods. First, the presence of the Egr-1 transcription factor was determined by Western blot analyses of cell pellets collected after the loading experiments (FIG. 4b). Secondly, the presence of secreted growth factors was determined by ELISA assay of tissue culture medium (FIG. 4c).

4.1.2 Conclusions:

These results show under conditions of bone loading that the transcription factor Egr-1 is produced in human osteosarcoma-derived osteoblast-like cells. Application of bone loading to human TE85 cells stimulates the production and secretion of growth factors, an example of which is PDGF B.

4.2 Transfection of CMV TGFβ-1 into MC3T3E1 and ROS Cells Followed by Human TGFβ-1 and Mouse VEGF ELISA Assays of Cell Culture Supernatents.

4.2.1 Materials:

(i) Transfection

Mouse Osteoblast cells (MC3T3E1) and rat Osteosarcoma cells (ROS17/2.8) were used seeded in 6 well plates.

MC3T3E1 cells were cultured in MEM-α, Minimum essential medium eagle, alpha modification (Sigma), 10% Foetal calf serum (Life Technologies), 1% L-glutamine (Life Technologies), 1% penicillin-streptomycin (Life Technologies).

ROS cells were cultured in F12 HAM, F-12 HAM with glutamine (Life Technologies), 10% Foetal calf serum (Life Technologies), 1% penicillin-streptomycin (Life Technologies).

Cells were transfected using Fugene (Boehringer Mannheim) with a CMV TGF-β1 expressing plasmid as described (Benn, S. I. et al, J. Clin. Invest., 98; 2894-2902, 1996). Transfection into cells was carried out as described:

1) A six well plate was prepared with 2×10⁵ cells per well and left overnight, until 50-70% confluent.

2) Next day 94 μl of Serum free media (SFM) and 6 μl of Fugene were added to each of 6 eppendorf tubes and left at room temperature for 5 min.

3) To 6 separate tubes, no DNA was added to 2 tubes, while 4 μg of CMV-TGF-β1 DNA was added to the remaining 4 tubes.

4) The Fugene/SFM mix from step 2) was added dropwise to the tubes from step 3), the tubes were flicked several times and then incubated for 15 min at room temperature.

5) The Fugene/SFM/DNA transfection mixes were added dropwise to their respective wells, while swirling the 6 well plate, the plate was incubated at 37° C. for 48 hrs.

6) Cell culture supernatents were aliquoted and stored at −20° C. The above protocol was performed for both MC3T3E1 cells and ROS cells. The presence of TGF-β1 and VEGF in the cell culture supernatant was detected by ELISA (R&D Systems) using a streptavidin-HRP based colour detection system.

4.2.2 Results:

Production and detection of TGF-β1 and VEGF following transfection of CMV-TGF-β1 is shown in ROS cells (FIG. 3) and MC3T3E1cells (FIG. 4). These data show that an Egr-1 target gene, in this example TGF-β1 have activate the production of VEGF.

4.3 Conclusion

Expression of Egr-1 and activation of Egr-1 target genes may synergistically activate VEGF.

### Example 5

Use of Egr-1 Transcription Factor to Promote Osteogenesis in Vivo

5.1 Rat Ectopic Bone Formation

Subcutaneous implantation of possible bone inducing compounds in rodents represents the most extensively studied biological assay system in current use (Wozney, J. M., Cell. Mol. Biol., 131-167, 1993). The use of a carrier matrix enhances the reproducibility and sensitivity of the bone induction response. In this assay system (Reddi, A. H. et al, Proc. Natl. Acad. Sci. USA, 69; 1601-1605, 1972; Sampath, T. K., ibid, 78; 7599-7603, 1981) the carrier matrix is derived from the diaphyseal portion of rat long bones that have been ground into particles of a particular size, subsequently demineralised and biological activity removed by guanidine extraction. The remaining carrier consists primarily of bone collagen with no osteoinductive capacity. The compound or substance to be assayed is then deposited onto the matrix by precipitation with alcohol, dialysis against water or lyophilisation. This matrix combination is then implanted into the subcutaneous tissues of the rat for a number of days (12 days in this experiment). The implants are then assayed histologically and biochemically for their ability to induce bone formation (Sampath, T. K. et al, Proc. Natl. Acad. Sci. USA, 80; 6591-6595, 1983; Sampath, T. K. et al, ibid, 84; 7109-7113, 1987; Wang, E. ibid, 85; 9484-9488; Wang, E. et al, ibid, 87; 2220-2224; Sampath, T. K. et al, J. Cell Biol., 98; 2192-2197, 1984).

5.1.1 Experimental Methods:

Prep Twenty male Sprague Dawley rats (age 42-49 days, weight 170-220 g) were randomly allocated to receive two implants inserted subcutaneously over the dorsal thorax under halothane anesthesia. The implants comprised one of four treatments:

Negative control—Carrier alone (demineralised guanidine extracted bone matrix DGBM)

CMV Egr 1 DNA; 500 ug on carrier DGBM

CMV Egr 1 DNA; 500 ug plus recombinant bone morphogenetic protein (BMP) 4; 5 ug on carrier DGBM, (BMP4 being used for its chemotactic effects)

Recombinant BMP4 Protein 5 ug on carrier DGBM

The day of insertion was regarded as day 0 and on day 12 post operatively all rats were killed using a schedule 1 approved method, the implants were removed, cleaned of soft tissue and divided into equal halves. One half was placed in 10% formalin for histological examination and the other half was frozen and stored at −20 degrees centigrade. This sample was then assayed for calcium content and alkaline phosphatase activity.

5.1.2 Preparation of Demineralised Rat Bone:

The diaphyseal shafts of the femora, tibiae and humeri of adult Sprague Dawley rats were removed, stripped of soft tissue and the marrow cavities irrigated with normal saline. The bone was then defatted by stirring in 100 ml Chloroform:Methanol (2:1} for 30 min. This step was repeated once prior to air drying in a drying oven. The bone shafts were then frozen in liquid nitrogen and pulverised in a CRC micromill. The resultant powder was sieved to leave a discreet particle size of 75-425 um and then demineralised in 0.5 HCl for 3 hours with constant stirring. The mixture was then centrifuged for 30 minutes at 19,0000 rpm (Kontron CentriksT124, Rotor A8.24) at 15 degrees centigrade. The pellet was resuspended in 100 ml water stirred for one hour and centrifuged. This step was then repeated. The pellet was then resuspended in 100 ml ethanol stirred for one hour and centrifuged. The ethanol was evaporated off and the sample resuspended in 4M guanidine hydrochloride/50 mM Tris, pH7.4 and stirred overnight. Further centrifugation was then carried out with the pellet resuspended in 50 ml water stirred for one hour and centrifuged. This step was repeated a further two times. The sample was then dried overnight in a drying oven. DNA was added to bone by mechanical mixing and lyophilisation.

5.2 Histological Examination

After initial fixation in formalin, the samples were embedded in methyl methacrylate and 1 μm sections cut and stained with Von Kossa and Toluidine Blue. Three nonadjacent sections from each implant were then evaluated by a consultant histopathologist blinded to the test substance and the scores averaged.

A standard scoring system for cartilage and bone was used;

+/−tentative identification of bone or cartilage

1. >10% each section new cartilage or bone

2. >25% each section new cartilage or bone

3. >50% each section new cartilage or bone

4. >75% each section new cartilage or bone

5. >80% each section new cartilage or bone

5.3 Biochemical Testing

The tissue was homogenized in 2 ml of ice cold 0.25 M sucrose-3mMNaHCO3. The homogenates were centrifuged at 12,000 g for 15 min at 2 degrees centigrade and the supernatants collected for enzyme assays. Alkaline phosphatase activity was determined using a colourimetric assay with p-nitrophenyl phosphate (PNP) as the substrate. After incubation of test samples with PNP at 37 degrees centigrade, optical density was determined at 405 nm in a standard micotitre plate reader.

5.4 Results.

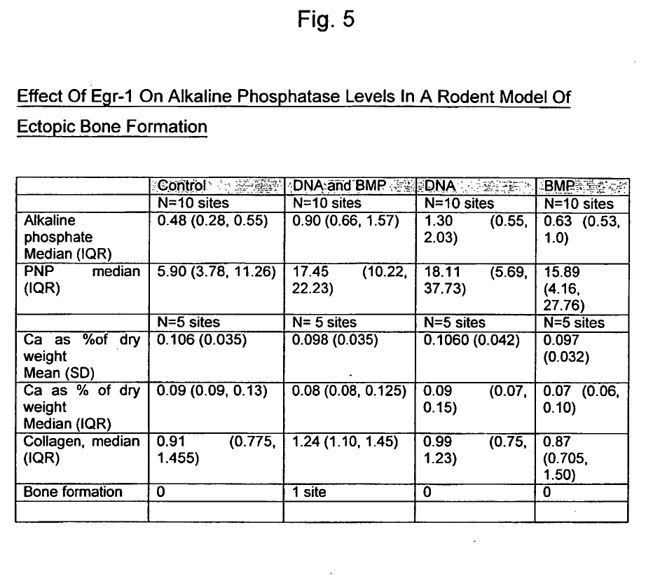

The results are presented in FIG. 5. The data was analysed treating the two implant sites for each rat as independent from one another. Medians and interquartile ranges (IQR) are presented because of the small numbers and the skewed distribution of the data. Kruskal Wallis tests have been carried out on the above variables and found that the alkaline phosphate levels differ significantly from one another.

Bone formation was positive for one implant in five implanted sites in only one group (CMV Egr-1 DNA/BMP). The initial experiment used a single timepoint for assay of 12 days, which was chosen to give early predictive results. At this timepoint levels of alkaline phosphatase activity are significantly elevated in CMV Egr-1 DNA and CMV Egr-1 DNA/BMP4 groups over controls. Such temporal increase in alkaline phosphatase activity is seen typically (as a precursor to bone formation) with substances such as BMP which stimulate bone formation rising to a peak at 10-15 days and falling thereafter. This represents the rise seen in the earliest phase of enchondral ossification. Calcium content does not show significant differences in the samples so far tested although early calcification has been observed in a number of histological samples in the CMV-Egr-1/BMP4 group. This may be explained due to the timescale of biopsy where calcification is only just starting.

5.5 Conclusion

Egr-1 increases alkaline phosphatase levels in a rodent model of ectopic bone formation and may promote localised bone formation.

### Example 6

Use of Egr-1 Transcription Factor to Promote Re-endothelialisation after Percutaneous Transluminal Coronary Angioplasty in Vitro

6.1 Methods

Human or porcine vascular smooth muscle cells (SMC; Clonetics) were thawed, maintained in medium and passaged until no later than passage 4 according to the manufacturer's instructions. SMC were transfected with an expression plasmid comprising the Egr-1 cDNA expressed from the CMV promoter Houston et al, Arterioscler. Thromb. Vasc. Biol., 19; 218-289, 1999). Egr-1 expressing DNA was transfected into SMC using Fugene (Boehringer Mannheim) after optimisation of SMC with the luciferase reporter vector pGL3 control (Promega) or Mirus Transit (Cambridge Biosciences) both transfection protocols used β-galactosidase as a normalising plasmid for transfection control.

6.2 Results

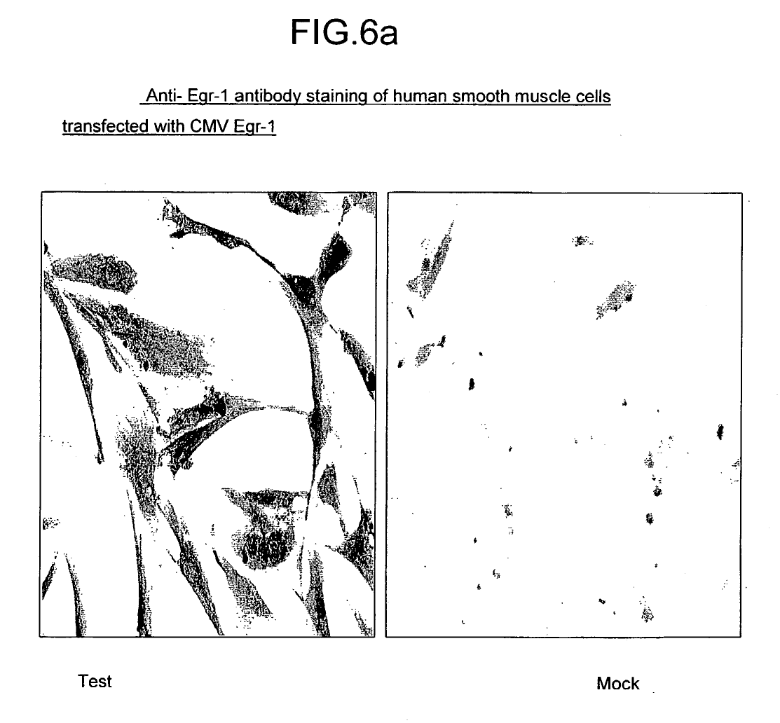

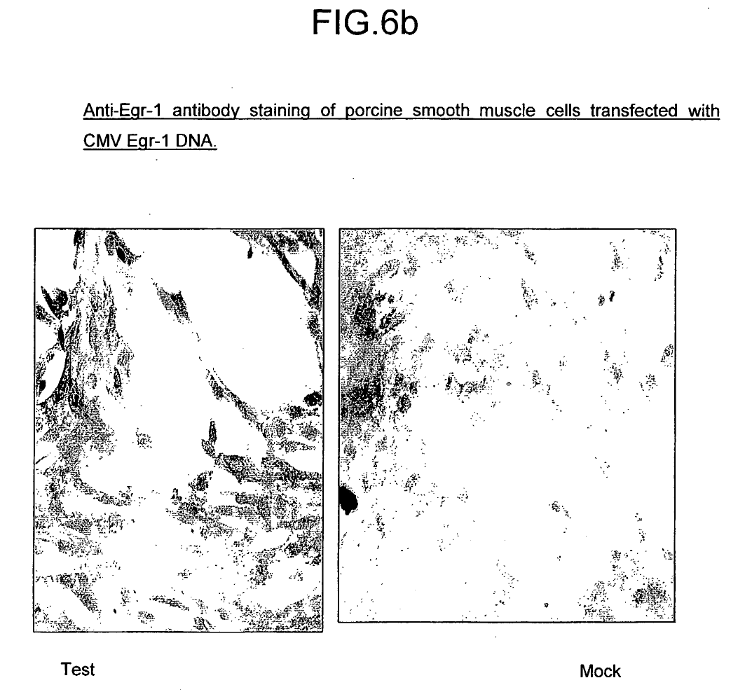

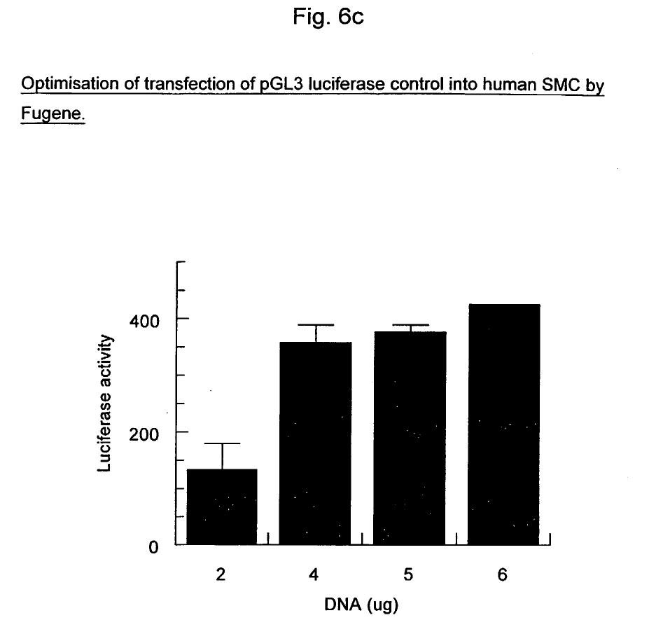

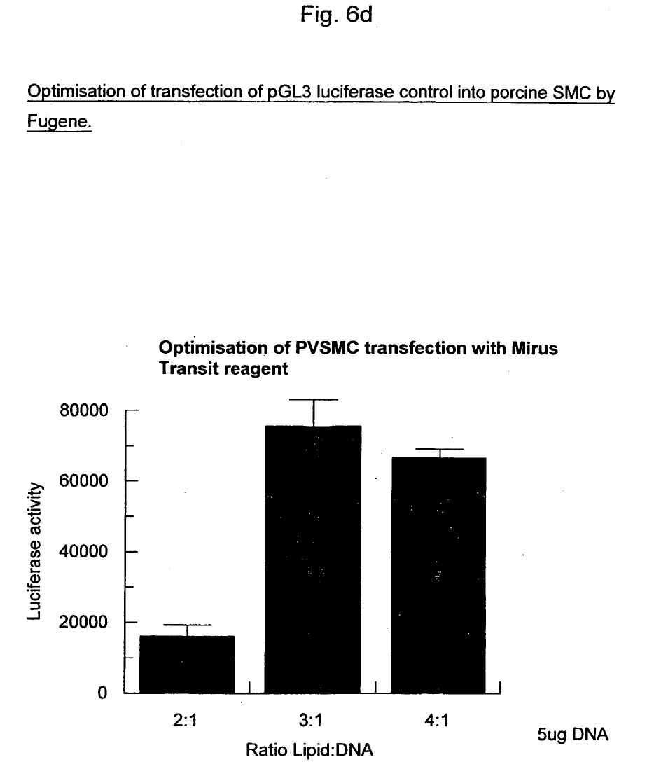

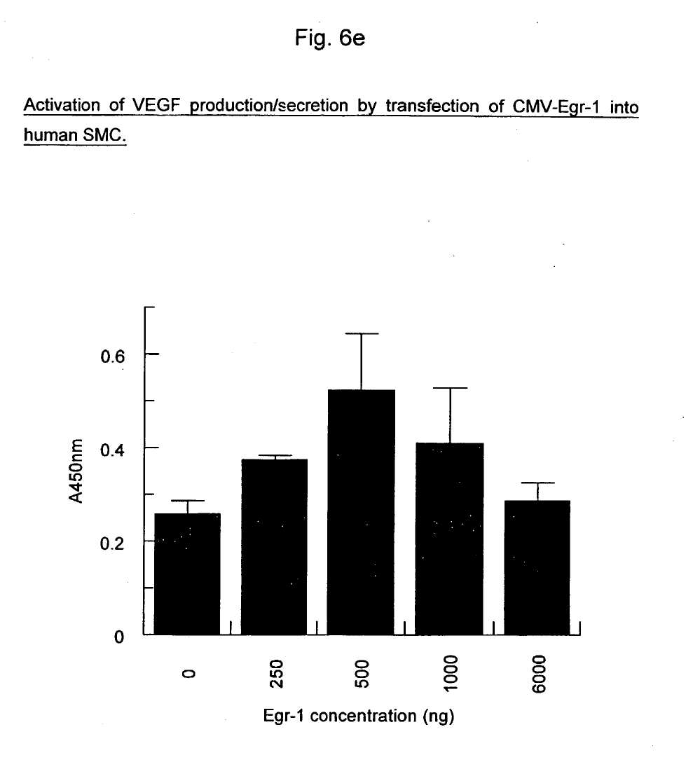

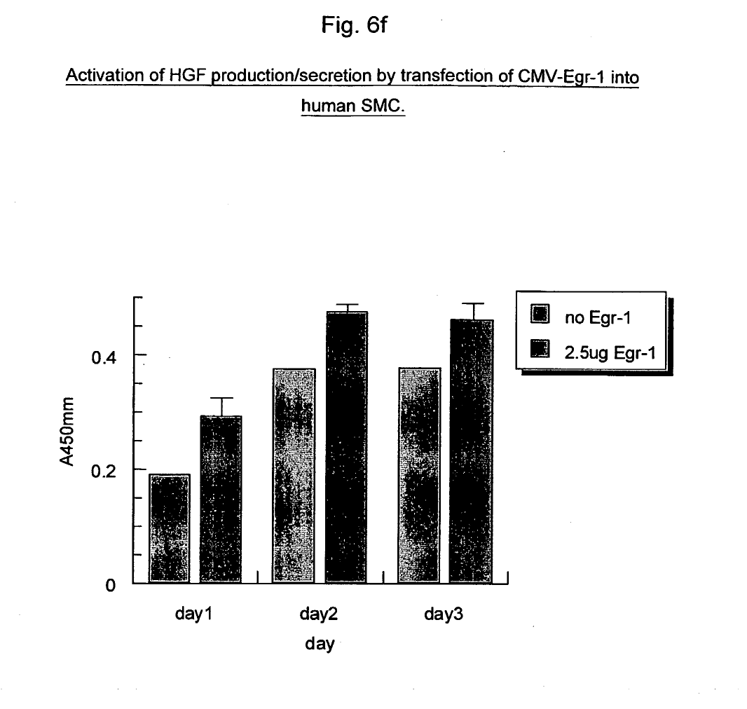

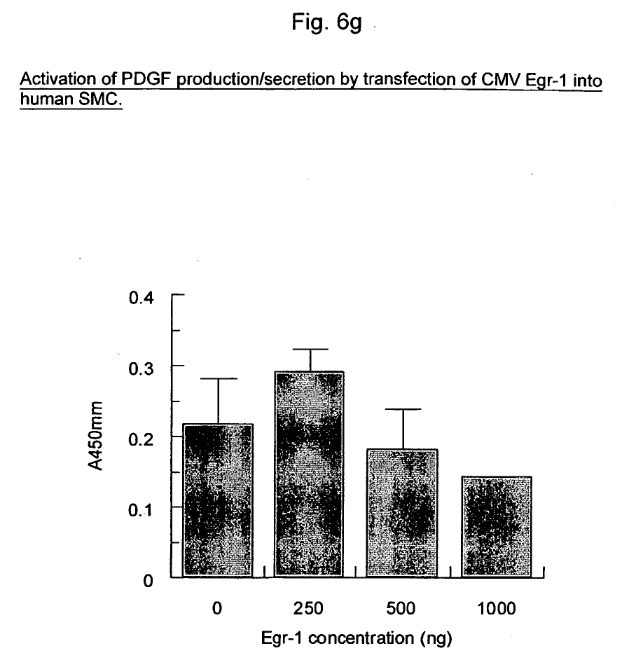

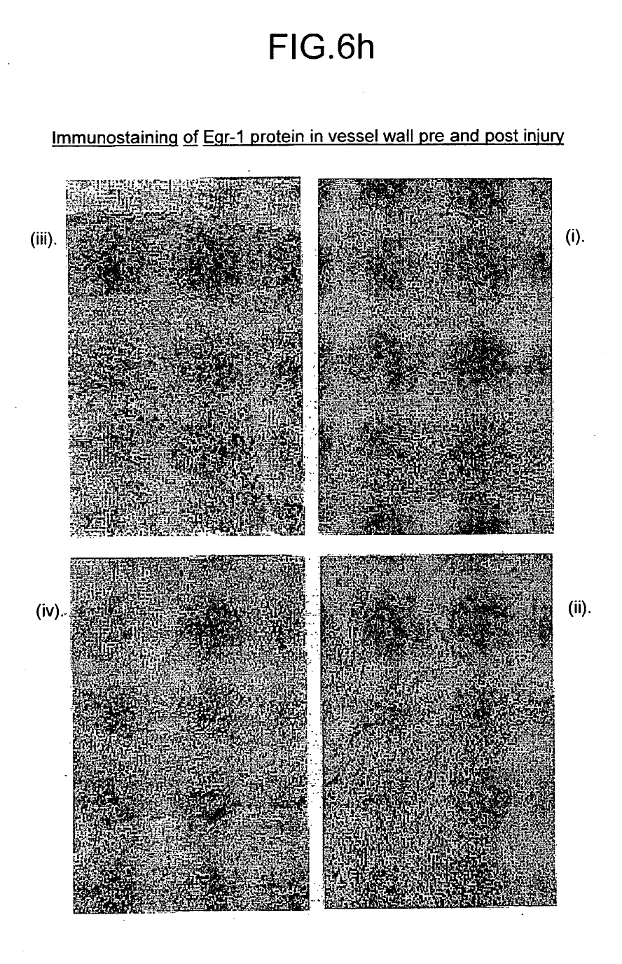

CMV Egr-1 DNA was transfected into human SMC and Egr-1 protein was detected by immunohistochemistry using a polyclonal antibody (Santa Cruz) and peroxidase detection (Sigma and Vector Laboratories). Human SMC transfected with CMV Egr-1 DNA (right hand panel) or mock transfected (left hand panel) are shown in FIG. 6a., and porcine SMC transfected with CMV Egr-1 DNA (right hand panel) or mock transfected (left hand panel) are shown in FIG. 6b. Egr-1 protein expression is detectable as brown staining. Optimisation of DNA transfection was achieved using Fugene 6 (for further in vitro characterisation, FIG. 6c.) and Mirus Transit (for subsequent in vivo studies, FIG. 6d.). From these data, 4 μg CMV-Egr-1 DNA was used routinely for growth factor activation experiments using a lipid:DNA ratio of 3:1. A lipid:DNA ratio of 3:1 was also used for in vivo gene delivery experiments.

Egr-1 activation of three growth factors was analysed by ELISA assay of cell supernatants. VEGF (FIG. 6e.), HGF (FIG. 6f.) and PDGF-AB (FIG. 6g.) production were all increased as a consequence of Egr-1 activation. There was a dose response of activation and an inverse dose response above a certain [Egr-1] DNA concentration as shown previously in Example 3.

6.3 Conclusion

Egr-1 protein is expressed in SMC following transfection of a CMV Egr-1 DNA. Transfection of Egr-1 increases the production/secretion of SMC derived PDGF, HGF and VEGF.

### Example 7

EGR-1 Promoter Sequence

The human Egr-1 promoter fragment spanning nt. −674 to +12 was synthesised by PCR in a reaction containing 0.5 μg of human placental genomic DNA as a template, 0.4 mM of dATP, dCTP, dGTP and dTTP, 25 pmoles of the forward primer (5′-GGC CAC GCG TCG TCG GTT CGC TCT CAC GGT CCC-3′, Mlu I restriction site is underlined), 25 pmoles of the reverse primer 5′-GCA GCT CGA GGC TGG ATC TCT CGC GAC TCC-3′ (Xho I site is underlined) and Vent DNA polymerase (NEB). The PCR fragment was cut with Mlu I and Xho I, agarose gel-purified and cloned between the Mlu I and Xho I sites in the multiple cloning site of the vector pGL3 basic (Promega).

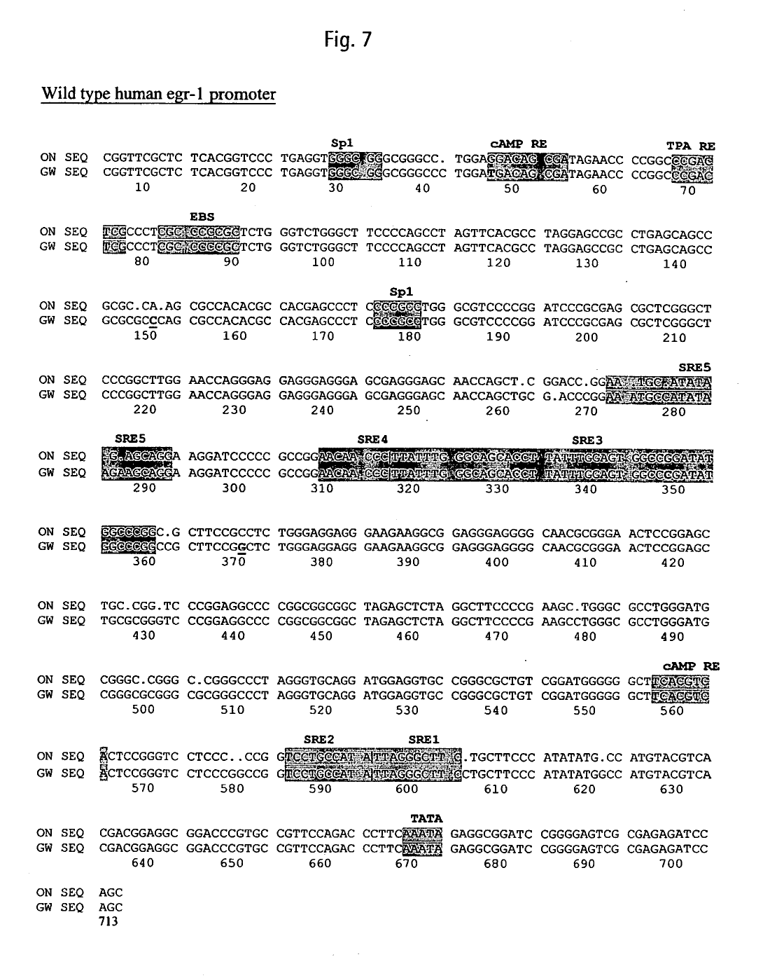

The full sequence has now been derived allowing completion of ‘gaps’ within the published sequence. This is shown in FIG. 7, where the complete sequence as derived by the inventors (GW SEQ) is compared with the previously published sequence (ON SEQ). This promoter sequence is functional and has been investigated in studies of shear stress on endothelial cells.

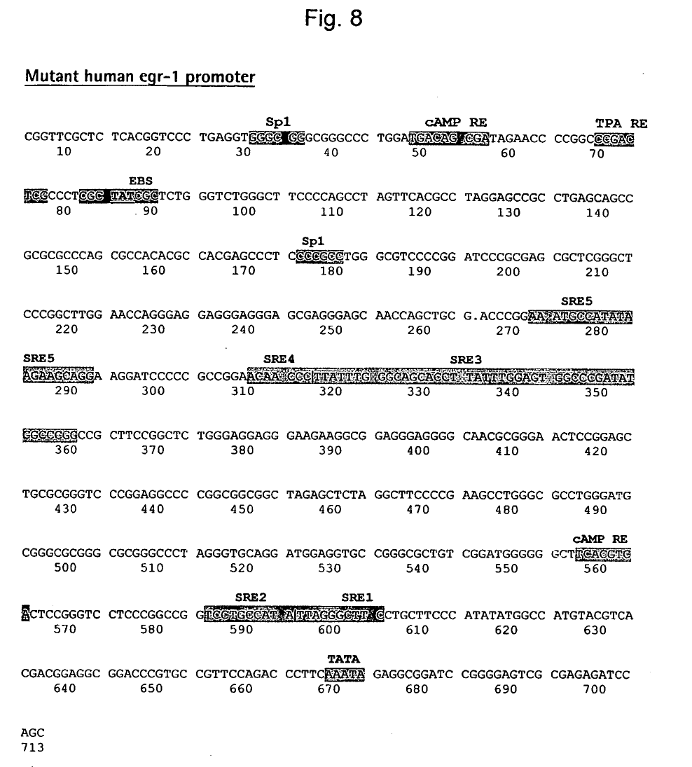

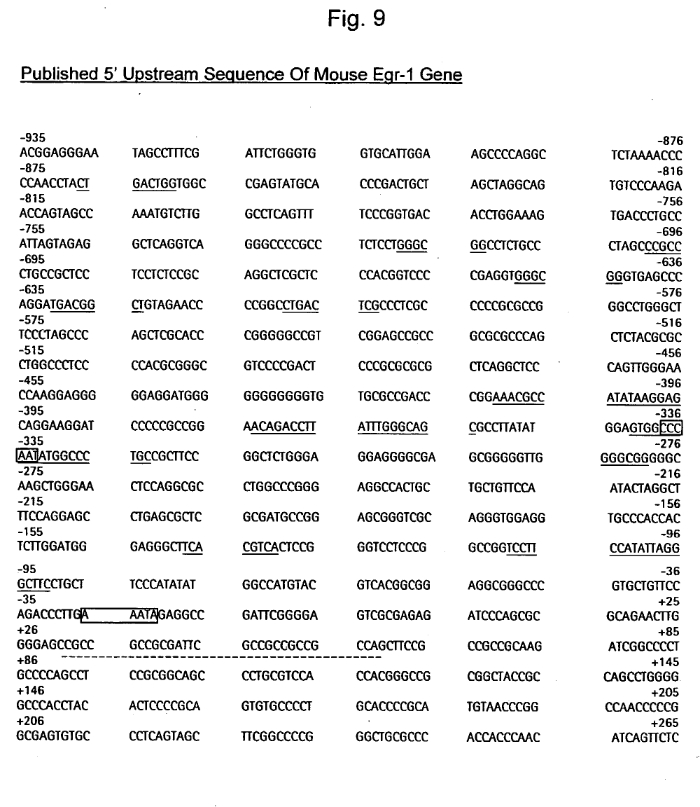

An important difference between the published sequence of the human Egr-1 promoter and the sequence which we describe (FIG. 8), lies in two previously unrecognised SREs. While the sequence of SRE 5 and SRE 1 as published do not bind serum response factor (SRF) and are not functional (Nurrish S J, Treisman R, Mol Cell Biol 1995, 15(8):4076-85), we have found that they are in agreement with the SRE consensus sequence (FIG. 7).

We have concentrated on SRE5. The novel SRE 5 with its associated Ets transcription factor binding sites was synthesised as a double stranded oligonucleotide and inserted into the Nhe I site upstream of a SV40 minimal promoter vector (pSV40).

SRE5 has the sequence:

AG GCTGCGACCCGGAAATGCCATATAAGGAGCAGGAAGGATCCCCCCGCCGG CGACGCTGGGCCTTTACGGTATATTCCTCGTCCTTCCTAGGGGGGCGGCC GA

The 2 Ets sites are bold, the SRE is underlined. The overhang AG is used to clone into the partially filled Nhe site of pGLE promoter.

The resulting reporter plasmid pSVSRE5 was transiently transfected into HeLa cells together with plasmids pFA-dbd (construct encoding the Gal4 DNA binding domain (dbd) or pFA-MEK1 (construct encoding a fusion protein of the Gal4 DNA binding domain (dbd) and the kinase domain of the MAP kinase kinase MEK1). The Gal4-MEK1 fusion protein is constitutively active and phosphorylates Elk1 and SRF, bound to SRE5.

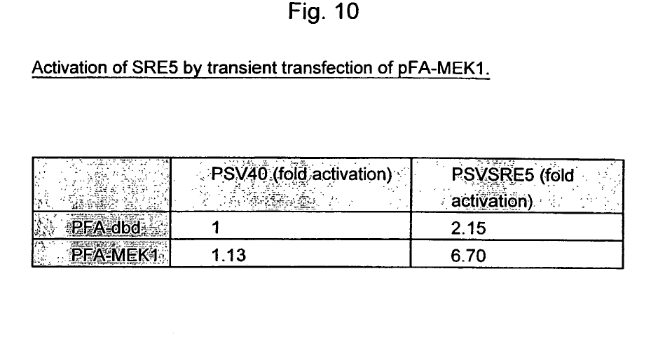

The results shown in FIG. 10 show that the isolated SRE5 sequence is activated 3-fold by the presence of MEK1, while the SV40 promoter shows only minimal activation.

The results indicate that the novel SRE5 is functional.

### Example 8

Cell-based Screen for Discovery of Compounds Stimulating the Activity or Production of Egr-1

Methods

1.1.1 Construction of Egr-1 Dependent Promoter Reporter Plasmids

A cell-based screen was established using transient transfection of promoter reporter plasmids. The sequence used was that of the tissue factor promoter which has been reported (Cui et al J. Biol. Chem. 271; 2731-2739). Two plasmid constructs were used; a wild type and mutant Egr-1 promoter reporter plasmid. Each plasmid was constructed using PCR amplification of human genomic DNA (Sigma) for 25 cycles and inserted into the BgIII and HindIII sites of the reporter gene pGL3 basic. The sequence of each plasmid is described below.

Plasmid one, hereafter referred to as EGRWT, which harbours wild type Egr-1 binding sites was prepared by PCR amplification using forward and reverse primers

Forward: 5′ ACGTACAGATCTCCCGGGCCGGGGGCGGGGAGTCGGG 3′;

Reverse: 5′ AGCTATAAGCTTGAGCTCGCAGTCTTGGGGAGCCGGT 3′.

Plasmid two, hereafter referred to as EGRMT, which harbours two mutated Egr-1 binding sites was constructed using oligonucleotides of sequence: 5′GATCTCCCGGGMTGGGGCGGGGAGTCGGGAGGAGCGMTGGGGCGGGC GAATGGGGCGGGCAGAGGCGCGGGAGAGCGCGCCGCCGGCCTTTATAGCG CGCGGGGCACCGGCTCCCCMGACTGCGAGCTCA 3′ and its complementary sequence.

Egr-1 has been shown to be stimulated by phorbol-myristate acetate as described (Khachigian et al, J. Biol. Chem., 270; 27679-27686, 1995). Thus PMA may act as a positive control compound for the screen.

1.1.2 Transient Transfection of Reporter Promoter Plasmids into HeLa Cells

Reporter plasmids EGRWT and EGRMT can be transiently transfected into HeLa cells using the Effectine liposome delivery method (Qiagen). HeLa cells can be cultured in DMEM supplemented with 10% foetal calf serum, 5% glutamine and 5% penicillin-streptomycin (all supplied from Gibco BRL). 10⁷ cells can be transfected with 40 μg of either reporter plasmid using Effectine reagent. Post-transfection, the cells can be trypsinised with 10% trypisn in PBS-EDTA (Gibco BRL) and seeded into 96 well micro-titre plates (Costar).

1.1.3 Compound Screening: Primary Assay

Each compound can be tested over a three log concentration range typically. 1 μM, 100 nm and 10 nm by addition to wells of the micro-titre plate. The appropriate amount of DMSO solvent can be used as the negative control. Luciferase activity can be detected 24 hr post compound addition using the Steady-Glo luciferase detection system (Promega) in a luminometer (Wallac). Positive hits can be independently verified. Specificity of the compound for Egr-1 stimulatory effects can be verified in a secondary screening comprising HeLa cells transiently transfected with reporter plasmid EGRMT.

1.1.4 Compound Screening: Secondary Assays

Compound hits from the primary and secondary screens of the primary assays can be further validated as follows: cells can be stained immuno-histochemically using an antibody raised against Egr-1 (Santa Cruz) as described in international patent application number PCT/GB99/01722. In addition, stimulation of growth factor secretion can be assayed by ELISA for both VEGF and PDGF-B as described in international patent application number PCT/GB99/02199.

Terminology

For the avoidance of doubt, certain terminology used in respect of the present invention is discussed in greater detail below. Similar terminology should be construed accordingly unless the context indicates otherwise.

“Egr-1”

The term Egr-1 is used herein to indicate a molecule having an Egr-1 activity. Preferred such molecules are identical in amino acid sequence to naturally occurring Egr-1 molecules (e.g. human Egr-1) or have substantial sequence identity therewith. Alternatively they may be fragments of naturally occurring molecules that retain an Egr-1 activity.

The amino acid sequence of Egr-1 is highly conserved between species, for example with 98% homology between rat and mouse.

The murine Egr-1 DNA sequence is known (Cell, 53 37-43 (1988)). The deduced amino acid sequence shows a long open reading frame with a stop codon (TAA) at position 1858. The deduced amino acid sequence predicts a polypeptide of 533 amino acids with a molecular weight of 56,596. The corresponding sequences from other species may be obtained by methods known in the art, for example by the screening of genomic or cDNA libraries using as probes oligonucleotide sequences based on or deriving from the murine Egr-1 sequence. Human Egr-1 is known to be located on chromosome 5, more precisely at 5q23-31 (Cell 53, 37-43). The sequence of the human Egr-1 cDNA is described in Nucleic Acids Research 18 p4283, 1990. The similarity between the mouse and human sequences is 87% and 94% at the nucleoside and amino acid levels, respectively.

“Egr-1 Activity”

This is a biological activity of Egr-1 (preferably of human Egr-1).

Such activities include any of the characteristics of Egr-1 listed herein or known to those skilled in the art, e.g.

1. Transcriptional activation of one or more of EGF, PDGF-A, bFGF, PDGF-B, TGF beta

2. Induction of PDGF-A, PDGF-B, TGF beta, bFGF, u-PA and IGF-2 expression.

3. The promotion of one or more of the following in an animal (e.g. rat) model of acute injury: angiogenesis, re-epithelialisation, collagen production, wound contraction.

4. Ectopic bone formation in an animal (e.g. rat) model

5. Promotion of bone healing in a rabbit model of radial osteotomy.

6. Activity in one or more of the treatments/medical uses disclosed herein

“Polypeptide”

This is used in a broad sense to indicate that a particular molecule comprises a plurality of amino acids joined together by peptide bonds. It therefore includes within its scope molecules that may sometimes be referred to as peptides, polypeptides or proteins.

Polypeptides may be provided in any appropriate form. They may be linear or non-linear (e.g. the ends may be joined together to provide a structure that is sometimes referred to as a “circular” or “endless” structure.

“Native Polypeptide”

This is a polypeptide found in nature in a given species of animal (e.g. in humans). It may be a wild-type polypeptide or a variant thereof that occurs in that species. It may be a polypeptide of the present invention. Preferably, it is in active form.

“Comprising” or “Having”

These terms covers not only anything consisting of a specified feature/characteristic, but also anything including that feature/characteristic, but also possessing one or more additional features/characteristics. Thus in the case of a nucleic acid/amino acid sequence comprising/having a given sequence, the sequence itself is covered, as are longer sequences.

“Substantially Pure Form” and “Isolated Form”

The term “substantially pure form” is used to indicate that a given component is present at a high level. The component is desirably the predominant component present in a composition. Preferably it is present at a level of more than 30%, of more than 50%, of more than 75%, of more than 90%, or even of more than 95%, said level being determined on a dry weight/dry weight basis with respect to the total composition under consideration. At very high levels (e.g. of more than 90%, of more than 95%, or of more than 99%), the component may be regarded as being in “isolated form”.

Biologically active substances of the present invention (including polypeptides, nucleic acid molecules, binding agents, moieties identified via screening, etc.) may be provided in a form that is substantially free of one or more contaminants with which the substance might otherwise be associated. Thus, for example they may be substantially free of one or more potentially contaminating polypeptides and/or nucleic acid molecules. They may be provided in a form that is substantially free of other cell components (e.g. of cell membranes, of cytoplasm, etc.). When a composition is substantially free of a given contaminant, the contaminant will be at a low level (e.g. at a level of less than 10%, less than 5%, or less than 1% on the dry weight/dry weight basis set out above)

“Sequence Identity”

For the purposes of the present invention, the percentage sequence identity between two amino acid or nucleotide sequences can be determined by using the algorithm of E. Meyers and W. Miller (CABIOS, 4:11-17 (1989)) which has been incorporated into the ALIGN program (Version 2.0.), using a PAM120 weight table, a gap length penalty of 12 and a gap penalty of 4.

Where high degrees of sequence identity are present there may be relatively few sequence differences. Thus, for example there may be less than 20, less than 10, or less than 5 differences.

“Treatment”

This includes any therapeutic applications that can benefit a human or non-human animal. The treatment of mammals is particularly preferred.

Thus, both human and veterinary treatments are within the scope of the present invention.

Non-human animals include farm animals, pets, wild animals, animals bred for racing or hunting, zoo and aquarium animals, etc. Particular non-human animals include mice, rats, guinea pigs, rabbits, sheep, goats, horses, cows, pigs, cats, dogs, birds, reptiles, fish, birds, monkeys and apes

Treatment may be in respect of an existing condition or it may be prophylactic. It may be of an adult, a juvenile, an infant or a foetus.

Where treatments are discussed, it will be appreciated that pharmaceutical compositions comprising the active agent can be provided and are within the scope of the present invention. The active agent/composition may be administered via any appropriate route of administration and at any appropriate dosage.

Disorders to be treated may be genetic in origin. Thus, they may arise due to one or more mutations that result in a deleterious effect—e.g. mutations in genes or in other regions. Mutations may result in excessive, insufficient, or aberrant expression of a gene product.

Disorders to be treated may also/alternatively arise due to environmental factors.

Treatments may be by via any appropriate method—e.g. via gene therapy techniques or via no gene therapy techniques (e.g. via administration to a patient of an active agent in a pharmaceutical composition)

NB. The foregoing comments in respect of treatments apply mutatis mutandis to medical uses.

“Treatment of Wounds”

This includes treatment of conditions associated with wounds, wound healing and associated conditions and therapy which promotes, augments or accelerates healing of tissues and includes the treatment of limb ulcerations in diabetes and peripheral arterial occlusive disease, post-operative scarring, burns, psoriasis, acceleration of tissue remodelling and bone repair and the promotion of angiogenesis, re-endothelialisation following percutaneous trans-luminal coronary angioplasty, inhibition of left ventricular cardiac hypertrophy, modulation of vessel wall calcification, and promotion of neuroregeneration. It further includes inhibition of fibrotic conditions, for example, pulmonary and liver fibrosis, and prevention of alopecia.

“Pharmaceutical Composition”

This is a composition that comprises or consists of a pharmaceutically active agent. It preferably includes a pharmaceutically acceptable carrier. This pharmaceutical composition will desirably be provided in a sterile form. It may be provided in unit dosage form, will generally be provided in a sealed container. A plurality of unit dosage forms may be provided.

Pharmaceutical compositions within the scope of the present invention may include one or more of the following: preserving agents, solubilising agents, stabilising agents, wetting agents, emulsifiers, sweeteners, colorants, odourants, salts (polypeptides of the present invention may themselves be provided in the form of a pharmaceutically acceptable salt), buffers, coating agents or antioxidants. They may also contain other therapeutically active agents in addition to polypeptides of the present invention.

Therapeutically active agents may themselves be provided in any suitable form—i.e. they may be used as such or may be used in the form of a pharmaceutically effective derivative. For example they may be used in the form of a pharmaceutically acceptable salt or hydrate. Pharmaceutically acceptable salts include alkali metal salts (e.g. sodium or potassium salts), alkaline earth metal salts (e.g. calcium or magnesium salts) aluminium salts, zinc salts, ammonium salts (e.g. tetra-alkyl ammonium salts), etc. Inorganic acid addition salts (e.g. hydrochlorides, sulphates, or phosphates) or organic acid addition salts (e.g. citrates, maleates, fumarates, succinates, lactates, propionates or tartrates) may be used.

Pharmaceutical compositions of the present invention may be provided in controlled release form. This can be achieved by providing a pharmaceutically active agent in association with a substance that degrades under physiological conditions in a predetermined manner. Degradation may be enzymatic or may be pH-dependent.

Pharmaceutical compositions may be deigned to pass across the blood brain barrier (BBB). For example, a carrier such as a fatty acid, inositol or cholesterol may be selected that is able to penetrate the BBB. The carrier may be a substance that enters the brain through a specific transport system in brain endothelial cells, such as insulin-like growth factor I or II,. The carrier may be coupled to the active agent or may contain/be in admixture with the active agent. Liposomes can be used to cross the BBB. WO91/04014 describes a liposome delivery system in which an active agent can be encapsulated/embedded and in which molecules that are normally transported across the BBB (e.g. insulin or insulin-like growth factor I or II) are present on the liposome outer surface. Liposome delivery systems are also discussed in U.S. Pat. No. 4,704,355.

“Route of Administration”

A pharmaceutical composition within the scope of the present invention may be adapted for administration by any appropriate route. For example, it may be administered by the oral (including buccal or sublingual), rectal, nasal, topical (including buccal, sublingual or transdermal), vaginal or parenteral (including subcutaneous, intramuscular, intravenous or intradermal) routes. Such a composition may be prepared by any method known in the art of pharmacy, for example by admixing one or more active ingredients with a suitable carrier.

Different drug delivery systems can be used to administer pharmaceutical compositions of the present invention, depending upon the desired route of administration. Drug delivery systems are described, for example, by Langer (Science 249:1527-1533 (1991)) and by Illum and Davis (Current Opinions in Biotechnology 2: 254-259 (1991)). Different routes of administration for drug delivery will now be considered in greater detail.

(i) Oral Administration

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, maize starch or derivatives thereof, stearic acid or salts thereof. Soft gelatine capsules may comprise vegetable oils, waxes, fats, semi-solid, or liquid polyols etc. Solutions and syrups may comprise water, polyols and sugars. For the preparation of suspensions oils (e.g. vegetable oils) may be used to provide oil-in-water or water-in-oil suspensions.

An active agent intended for oral administration may be coated with or admixed with a material that delays disintegration and/or absorption of the active agent in the gastrointestinal tract (e.g. glyceryl monostearate or glyceryl distearate may be used). Thus the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

(ii) Transdermal Administration

Pharmaceutical compositions adapted for transdermal administration may be provided as discrete patches intended to remain in intimate contact with the epidermis of the recipient for a prolonged period of time. For example, the active ingredient may be delivered from the patch by iontophoresis. (Iontophoresis is described in Pharmaceutical Research, 3(6):318 (1986).)

(iii) Topical Administration

Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. Here the active ingredient can be dissolved or suspended in a suitable carrier, e.g. in an aqueous solvent. Pharmaceutical compositions adapted for topical administration in the mouth include lozenges, pastilles and mouthwashes.

(iv) Rectal Administration

Pharmaceutical compositions adapted for rectal administration may be provided as suppositories or enemas.

(v) Nasal Administration

Pharmaceutical compositions adapted for nasal administration may use solid carriers—e.g. powders (preferably having a particle size in the range of 20 to 500 microns). Powders can be administered in the manner in which snuff is taken, i.e. by rapid inhalation through the nose from a container of powder held close to the nose. Compositions adopted for nasal administration may alternatively use liquid carriers—e.g. include nasal sprays or nasal drops. These may comprise aqueous or oil solutions of the active ingredient.

Compositions for administration by inhalation may be supplied in specially adapted devices—e.g. in pressurised aerosols, nebulizers or insufflators. These devices can be constructed so as to provide predetermined dosages of the active ingredient.

(vi) Vaginal Administration

Pharmaceutical compositions adapted for vaginal administration may be provided as pessaries, tampons, creams, gels, pastes, foams or spray formulations.

(vii) Parenteral Administration

Pharmaceutical compositions adapted for parenteral administration include aqueous and non-aqueous sterile injectable solutions or suspensions. These may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilised) condition requiring only the addition of a sterile liquid carrier, e.g. sterile water for injections, immediately prior to use. Extemporaneous injection solutions and suspensions may be prepared from sterile powders, granules and tablets.

From the above description it will be appreciated that compositions of the present invention can be formulated in many different ways. However preferred compositions of the present invention are aerosols, creams or gels.

“Dosages”

Dosages of a polypeptide of the present invention can vary between wide limits, depending upon the nature of the treatment, the age and condition of the individual to be treated, etc. and a physician will ultimately determine appropriate dosages to be used.

However, without being bound by any particular dosages, a daily dosage of a polypeptide of the present invention of from 1 μg to 1 mg/kg body weight may be suitable. The dosage may be repeated as often as appropriate. If side effects develop, the amount and/or frequency of the dosage can be reduced, in accordance with good clinical practice.

“Binding Agents”

Various binding agents can be used in the present invention.

One type of binding agent is an antibody. Antibodies for use in the present invention may be monoclonal or polyclonal.

Polyclonal antibodies can be raised by stimulating their production in a suitable animal host (e.g. a mouse, rat, guinea pig, rabbit, sheep, goat or monkey) when a polypeptide is injected into the animal. If desired, an adjuvant may be administered together with a polypeptide of the present invention. Well-known adjuvants include Freund's adjuvant (complete or incomplete) and aluminium hydroxide. The antibodies can then be purified by virtue of their binding to the polypeptide.

Monoclonal antibodies can be produced from hybridomas. These can be formed by fusing together myeloma cells and spleen cells that produce the desired antibody in order to form an immortal cell line. Thus the well-known Kohler & Milstein technique (Nature 256 (1975)) or subsequent variations upon this technique can be used.

Techniques for producing monoclonal and polyclonal antibodies that bind to a particular polypeptide are now well developed in the art. They are discussed in standard immunology textbooks—e.g. in Roitt et al, Immunology second edition (1989), Churchill Livingstone, London. Antibodies can be purified by adsorption to staphlylococcal protein A. The staphlylococcal protein will usually be coupled to a solid support, such as Sepharose beads. This can be done via cyanogen bromide coupling. Antibodies bind to protein A chiefly by hydrophobic interactions, which can be disrupted when desired so as to elute the antibodies (e.g. via transient exposure to low pH).

More recently, techniques such as phage display have been used to express antibodies. These techniques are becoming increasingly popular and are described for example by M J Geisow in Tibtech 10, 75-76 and by D. Chiswell et al in Tibtech 10, 8-84, (1992). They can be used to express antibodies recognising desired epitopes.

The above discussion focuses on whole antibodies. However the other moieties can be used—e.g. antibody fragments and synthetic constructs. Examples of antibody fragments and synthetic constructs are given by Dougall et al in Tibtech 12,372-379 (September 1994).

Antibody fragments include, for example, Fab, F(ab′)₂ and Fv fragments. Fab fragments (These are discussed in Roitt et al [supra]). Fv fragments can be modified to produce a synthetic construct known as a single chain Fv (scFv) molecule. This includes a peptide linker covalently joining Vₕ and Vₗregions, which contributes to the stability of the molecule. Other synthetic constructs that can be used include CDR peptides. These are synthetic peptides comprising antigen-binding determinants. Peptide mimetics may also be used. These molecules are usually conformationally restricted organic rings that mimic the structure of a CDR loop and that include antigen-interactive side chains.

Synthetic constructs include chimaeric molecules. Thus, for example, humanised (or primatised) antibodies are within the scope of the present invention. An example of a humanised antibody is an antibody having human framework regions, but rodent hypervariable regions. Ways of producing chimaeric antibodies are discussed for example by Morrison et al in PNAS, 81, 6851-6855 (1984), by Takeda et al in Nature. 314, 452-454 (1985) and by Cunningham et al in Tibtech 10, 112-113 (1992).

Synthetic constructs also include molecules comprising an additional moiety that provides the molecule with some desirable property in addition to antigen binding. For example the moiety may be a label (e.g. a fluorescent or radioactive label). Alternatively, it may be a pharmaceutically active agent.

A further type of binding agent that can be used in the present invention is a lectin. Lectins are carbohydrate binding proteins of non-immune (e.g. plant)origin (see e.g. the discussion of lectins by Deutscher in Methods in Enzymology, Guide to Protein Purification, 182 (1990).) Different lectins can be used to select particular glycoproteins based upon the presence of particular carbohydrate moieties (e.g. sialic acid, galactose, mannose, fucose, N-acetyl glucosamine, N-acetyl galactosamine, etc). In some cases a plurality of different lectins may be used—e.g. if a glycoprotein is known to include three different sugars, then three different lectins may be used to purify it. They may be used sequentially (e.g. in sequential affinity columns).

General Remarks

The description of the invention provided herein is merely illustrative thereof and it should therefore be appreciated that various variations and modifications can be made without departing from the spirit or scope of the invention as set forth in the accompanying claims.

Substances described herein (including nucleic acid molecules, vectors, polypeptides, moieties identified by screening, antibodies, etc.) may be provided in substantially pure form or in isolated form. However, they are not limited to being provided in such forms.

Where features are described in connection with particular aspects of the present invention, they shall be deemed to apply mutatis mutandis to other aspects of the invention, unless the context indicates otherwise.

All documents cited herein are hereby expressly incorporated by reference.

1. A method of screening comprising providing an expression system that is regulated by Egr-1 and analysing the effect of a moiety on expression from said system.

2. A method according to claim 1, wherein the expression system comprises a nucleic acid molecule having an Egr-1 binding site.

3. A method according to claim 2, wherein the expression system comprises a nucleic acid molecule having a plurality of Egr-1 binding sites.

4. A method according to any preceding claims, wherein said expression system comprises an Egr-1 dependent promoter.

5. A method according to any preceding claim, wherein expression of the gene product of a reporter gene is analysed.

6. A method according to any preceding claim, wherein said expression is a cell-based expression system.

7. A method according to any preceding claim, wherein expression is from an autosome (e.g. a plasmid).

8. A method according to any preceding claim, further comprising a control step whereby expression from the expression system is compared with expression from a system that is not regulated by Egr-1 or that is regulated to a lesser extent by Egr-1.

9. A moiety identified by a method of screening according to any preceding claim

10. A moiety according to claim 9 that increases Egr-1 activity or expression.

11. A moiety according to claim 9 that decreases Egr-1 activity or expression.

12. The use of a moiety that increases Egr-1 activity or expression in the manufacture of a medicament for treating wounds or associated conditions.

13. The use of a moiety that decreases Egr-1 activity or expression in the manufacture of a medicament for treating wounds or conditions involving cell proliferation.

14. The use of a moiety that increases or decreases Egr-1 expression in a drug development program.

15. The use according to any of claims 12 to 13, wherein the moiety is a moiety according to claim 9.

16. A drug developed from a drug development program as described in claim 14.

17. A pharmaceutical composition comprising a moiety according to any of claims 9 to 11, said composition optionally including a pharmaceutically acceptable carrier.

18. An expression system suitable for performing screening according to any of claims 1 to 8.

19. A method for identifying compounds which will be useful for the treatment of wounds comprising the step of determining whether the compounds activate Egr-1.

20. A method for treating wounds comprising administering a therapeutically effective amount of a compound identified by the method of claim 19.
